SEQUENCE LISTING (4 sequences)

SEQ ID NO: 1 - protein, 895 aa, Homo sapiens
MRMSVGLSLLLPLWGRTFLLLLSVVMAQSHWPSEPSEAVRDWENQLEASMHSVLSDLHEAVPTVVGIPDGTAVVGRSFRVTIPTDLIASSGDIIKVSAAGKEALPSWLHWDSQSHTLEGLPLDTDKGVHYISVSATRLGANGSHIPQTSSVFSIEVYPEDHSDLQSVRTASPDPGEVVSSACAADEPVTVLTVILDADLTKMTPKQRIDLLHRMRSFSEVELHNMKLVPVVNNRLFDMSAFMAGPGNPKKVVENGALLSWKLGCSLNQNSVPDIHGVEAPAREGAMSAQLGYPVVGWHIANKKPPLPKRVRRQIHATPTPVTAIGPPTTAIQEPPSRIVPTPTSPAIAPPTETMAPPVRDPVPGKPTVTIRTRGAIIQTPTLGPIQPTRVSEAGTTVPGQIRPTMTIPGYVEPTAVATPPTTTTKKPRVSTPKPATPSTDSTTTTTRRPTKKPRTPRPVPRVTTKVSITRLETASPPTRIRTTTSGVPRGGEPNQRPELKNHIDRVDAWVGTYFEVKIPSDTFYDHEDTTTDKLKLTLKLREQQLVGEKSWVQFNSNSQLMYGLPDSSHVGKHEYFMHATDKGGLSAVDAFEIHVHRRPQGDRAPARFKAKFVGDPALVLNDIHKKIALVKKLAFAFGDRNCSTITLQNITRGSIVVEWTNNTLPLEPCPKEQIAGLSRRIAEDDGKPRPAFSNALEPDFKATSITVTGSGSCRHLQFIPVVPPRRVPSEAPPTEVPDRDPEKSSEDDVYLHTVIPAVVVAAILLIAGIIAMICYRKKRKGKLTLEDQATFIKKGVPIIFADELDDSKPPPSSSMPLILQEEKAPLPPPEYPNQSVPETTPLNQDTMGEYTPLRDEDPNAPPYQPPPPFTVPMEGKGSRPKNMTPYRSPPPYVPP

SEQ ID NO: 2 - protein, 3075 aa, Homo sapiens
MRGGVLLVLLLCVAAQCRQRGLFPAILNLASNAHISTNATCGEKGPEMFCKLVEHVPGRPVRNPQCRICDGNSANPRERHPISHAIDGTNNWWQSPSIQNGREYHWVTITLDLRQVFQVAYVIIKAANAPRPGNWILERSLDGTTFSPWQYYAVSDSECLSRYNITPRRGPPTYRADDEVICTSYYSRLVPLEHGEIHTSLINGRPSADDLSPKLLEFTSARYIRLRLQRIRTLNADLMTLSHREPKELDPIVTRRYYYSIKDISVGGMCICYGHASSCPWDETTKKLQCQCEHNTCGESCNRCCPGYHQQPWRPGTVSSGNTCEACNCHNKAKDCYYDESVAKQKKSLNTAGQFRGGGVCINCLQNTMGINCETCIDGYYRPHKVSPYEDEPCRPCNCDPVGSLSSVCIKDDLHSDLHNGKQPGQCPCKEGYTGEKCDRCQLGYKDYPTCVSCGCNPVGSASDEPCTGPCVCKENVEGKACDRCKPGFYNLKEKNPRGCSECFCFGVSDVCSSLSWPVGQVNSMSGWLVTDLISPRKIPSQQDALGGRHQVSINNTAVMQRLAPKYYWAAPEAYLGNKLTAFGGFLKYTVSYDIPVETVDSNLMSHADVIIKGNGLTLSTQAEGLSLQPYEEYLNVVRLVPENFQDFHSKRQIDRDQLMTVLANVTHLLIRANYNSAKMALYRLESVSLDIASSNAIDLVVAADVEHCECPQGYTGTSCESCLSGYYRVDGILFGGICQPCECHGHAAECNVHGVCIACAHNTTGVHCEQCLPGFYGEPSRGTPGDCQPCACPLTIASNNFSPTCHLNDGDEVVCDWCAPGYSGAWCERCADGYYGNPTVPGESCVPCDCSGNVDPSEAGHCDSVTGECLKCLGNTDGAHCERCADGFYGDAVTAKNCRACECHVKGSHSAVCHLETGLCDCKPNVTGQQCDQCLHGYYGLDSGHGCRPCNCSVAGSVSDGCTDEGQCHCVPGVAGKRCDRCAHGFYAYQDGSCTPCDCPHTQNTCDPETGECVCPPHTQGVKCEECEDGHWGYDAEVGCQACNCSLVGSTHHRCDVVTGHCQCKSKFGGRACDQCSLGYRDFPDCVPCDCDLRGTSGDACNLEQGLCGCVEETGACPCKENVFGPQCNECREGTFALRADNPLGCSPCFCSGLSHLCSELEDYVRTPVTLGSDQPLLRVVSQSNLRGTTEGVYYQAPDFLLDAATVRQHIRAEPFYWRLPQQFQGDQLMAYGGKLKYSVAFYSLDGVGTSNFEPQVLIKGGRIRKQVIYMDAPAPENGVRQEQEVAMRENFWKYFNSVSEKPVTREDFMSVLSDIEYILIKASYGQGLQQSRISDISMEVGRKAEKLHPEEEVASLLENCVCPPGTVGFSCQDCAPGYHRGKLPAGSDRGPRPLVAPCVPCSCNNHSDTCDPNTGKCLNCGDNTAGDHCDVCTSGYYGKVTGSASDCALCACPHSPPASFSPTCVLEGDHDFRCDACLLGYEGKHCERCSSSYYGNPQTPGGSCQKCDCNPHGSVHGDCDRTSGQCVCRLGASGLRCDECEPRHILMETDCVSCDDECVGVLLNDLDEIGDAVLSLNLTGIIPVPYGILSNLENTTKYLQESLLKENMQKDLGKIKLEGVAEETDNLQKKLTRMLASTQKVNRATERIFKESQDLAIAIERLQMSITEIMEKTTLNQTLDEDFLLPNSTLQNMQQNGTSLLEIMQIRDFTQLHQNATLELKAAEDLLSQIQENYQKPLEELEVLKEAASHVLSKHNNELKAAEALVREAEAKMQESNHLLLMVNANLREFSDKKLHVQEEQNLTSELIVQGRGLIDAAAAQTDAVQDALEHLEDHQDKLLLWSAKIRHHIDDLVMHMSQRNAVDLVYRAEDHAAEFQRLADVLYSGLENIRNVSLNATSAAYVHYNIQSLIEESEELARDAHRTVTETSLLSESLVSNGKAAVQRSSRFLKEGNNLSRKLPGIALELSELRNKTNRFQENAVEITRQTNESLLILRAIPKGIRDKGAKTKELATSASQSAVSTLRDVAGLSQELLNTSASLSRVNTTLRETHQLLQDSTMATLLAGRKVKDVEIQANLLFDRLKPLKMLEENLSRNLSEIKLLISQARKQAASIKVAVSADRDCIRAYQPQISSTNYNTLTLNVKTQEPDNLLFYLGSSTASDFLAVEMRRGRVAFLWDLGSGSTRLEFPDFPIDDNRWHSIHVARFGNIGSLSVKEMSSNQKSPTKTSKSPGTANVLDVNNSTLMFVGGLGGQIKKSPAVKVTHFKGCLGEAFLNGKSIGLWNYIEREGKCRGCFGSSQNEDPSFHFDGSGYSVVEKSLPATVTQIIMLFNTFSPNGLLLYLGSYGTKDFLSIELFRGRVKVMTDLGSGPITLLTDRRYNNGTWYKIAFQRNRKQGVLAVIDAYNTSNKETKQGETPGASSDLNRLDKDPIYVGGLPRSRVVRRGVTTKSFVGCIKNLEISRSTFDLLRNSYGVRKGCLLEPIRSVSFLKGGYIELPPKSLSPESEWLVTFATTNSSGIILAALGGDVEKRGDREEAHVPFFSVMLIGGNIEVHVNPGDGTGLRKALLHAPTGTCSDGQAHSISLVRNRRIITVQLDENNPVEMKLGTLVESRTINVSNLYVGGIPEGEGTSLLTMRRSFHGCIKNLIFNLELLDFNSAVGHEQVDLDTCWLSERPKLAPDAEDSKLLPEPRAFPEQCVVDAALEYVPGAHQFGLTQNSHFILPFNQSAVRKKLSVELSIRTFASSGLIYYMAHQNQADYAVLQLHGGRLHFMFDLGKGRTKVSHPALLSDGKWHTVKTDYVKRKGFITVDGRESPMVTVVGDGTMLDVEGLFYLGGLPSQYQARKIGNITHSIPACIGDVTVNSKQLDKDSPVSAFTVNRCYAVAQEGTYFDGSGYAALVKEGYKVQSDVNITLEFRTSSQNGVLLGISTAKVDAIGLELVDGKVLFHVNNGAGRITAAYEPKTATVLCDGKWHTLQANKSKHRITLIVDGNAVGAESPHTQSTSVDTNNPIYVGGYPAGVKQKCLRSQTSFRGCLRKLALIKSPQVQSFDFSRAFELHGVFLHSCPGTES

SEQ ID NO: 3 - protein, 376 aa, Homo sapiens
THGPCAAESEPALLIGSKQFGLSRNSHIAIAFDDTKVKNRLTIELEVRTEAESGLLFYMARINHADFATVQLRNGLPYFSYDLGSGDTHTMIPTKINDGQWHKIKIMRSKQEGILYVDGASNRTISPKKADILDVVGMLYVGGLPINYTTRRIGPVTYSIDGCVRNLHMAEAPADLEQPTSSFHVGTCFANAQRGTYFDGTGFAKAVGGFKVGLDLLVEFEFRTTTTTGVLLGISSQKMDGMGIEMIDEKLMFHVDNGAGRFTAVYDAGVPGHLCDGQWHKVTANKIKHRIELTVDGNQVEAQSPNPASTSADTNDPVFVGGFPDDLKQFGLTTSIPFRGCIRSLKLTKGTGKPLEVNFAKALELRGVQPVSCPAN

SEQ ID NO: 4 - protein, 379 aa, Homo sapiens
QPELCAVDTAPGYVAGAHQFGLSQNSHLVLPLNQSDVRKRLQVQLSIRTFASSGLIYYVAHQNQMDYATLQLQEGRLHFMFDLGKGRTKVSHPALLSDGKWHTVKTEYIKRKAFMTVDGQESPSVTVVGNATTLDVERKLYLGGLPSHYRARNIGTITHSIPACIGEIMVNGQQLDKDRPLSASAVDRCYVVAQEGTFFEGSGYAALVKEGYKVRLDLNITLEFRTTSKNGVLLGISSAKVDAIGLEIVDGKVLFHVNNGAGRITATYQPRAARALCDGKWHTLQAHKSKHRIVLTVDGNSVRAESPHTHSTSADTNDPIYVGGYPAHIKQNCLSSRASFRGCVRNLRLSRGSQVQSLDLSRAFDLQGVFPHSCPGPEP

(19) United States
(12) Patent Application Publication
(10) Pub. No.: US 2015/0374845 A1
(43) Pub. Date: Dec. 31, 2015

(54) COMPOUNDS THAT BIND DYSTROGLYCAN AND USES THEREOF

(71) Applicants: John L. Muschler, Lake Oswego, OR (US); James Korkola, Lake Oswego, OR (US)

(72) Inventors: John L. Muschler, Lake Oswego, OR (US); James Korkola, Lake Oswego, OR (US)

(73) Assignee: OREGON HEALTH & SCIENCE UNIVERSITY, PORTLAND, OR (US)

(21) Appl. No.: 14/751,631

(22) Filed: Jun. 26, 2015

Related U.S. Application Data
(60) Provisional application No. 62/018,440, filed on Jun. 27, 2014.

(51) Int. Cl. A61K 47/48; A61K 31/537; G01N 33/574; C07K 14/78

(52) U.S. Cl. CPC A61K 47/48292; C07K 14/78; A61K 31/537; G01N 33/57492; G01N 2400/00

(57) ABSTRACT

Disclosed herein are methods and compositions involved in identifying cells that lack apico-basal polarity as well as methods and compositions involved in selectively delivering payload molecules to cells that lack apico-basal polarity, and methods of selecting test compounds that restore apico-basal polarity.

### FIELD

Generally, the field is methods and compositions used in identifying, treating, or eliminating cells that have lost apico-basal polarity. More specifically, the field is methods and reagents used in identifying, treating, or eliminating cells that lack or have lost apico-basal polarity using agents that bind to dystroglycan.

### BACKGROUND

Basement membranes (BMs) are critical regulators of tissue architecture and function, and, like all extracellular matrices (ECMs), are subject to dynamic remodeling during development, homeostasis, and tissue repair (Streuli C, Curr Opin Cell Biol 11, 634-640 (1999); Yurchenco P D, Cold Spring Harb Perspect Biol 3, (2011); both of which are incorporated by reference herein). Correspondingly, perturbation of cell-basement membrane interactions contributes to the progression of a wide range of human diseases including skin blistering diseases, muscular dystrophies, neuro-developmental defects, and cancers (Akhavan A et al, Cancer Res 72, 2578-2588 (2012); Barresi R and Campbell K P, J Cell Sci 119, 199-207 (2006); Domogatskaya A et al, Ann Rev Cell Dev Biol 28, 523-553 (2012); Yurchenco P D and Patton B L, Curr Pharm Des 15, 1277-1294 (2009); all of which are incorporated by reference herein). These perturbations are most often attributed to altered basement membrane receptor expression or function, altered synthesis of basement membrane proteins, or remodeling of basement membrane proteins by proteases (Akhavan et al, 2012 supra; Rowe R G and Weiss S J, Trends Cell Biol 18, 560-574 (2008); incorporated by reference herein). However, the many changes in cell-basement membrane communication that contribute to the progression of diseases are not fully understood and other previously unrecognized regulatory factors may also be involved (Rowe and Weiss, 2008 supra).

The internalization and endocytic trafficking of cell membrane and extracellular components are essential and integral functions regulating interactions between cells and their microenvironment (Polo S and Di Fiore P P, Cell 124, 897-900 (2006); Scita G and Di Fiore P P, Nature 463, 464-473 (2010); both of which are incorporated by reference herein). Endocytosis orchestrates cell-microenvironment interactions through multiple mechanisms, including the turnover of extracellular ligands and receptors, their recycling to the cell surface, and the spatio-temporal control of signaling events within the cell (Polo and Di Fiore, 2006 supra; Scita and Di Fiore, 2010 supra; Sorkin A and von Zastrow M, Nat Rev Mol Cell Biol 10, 609-622 (2009); incorporated by reference herein. The endocytosis of some ECM components, such as collagen I and fibronectin, has been investigated and demonstrated to regulate both matrix degradation and deposition in conjunction with β1 integrins (Shi F and Sottile J, J Cell Sci 121, 2360-2371 (2008); Sottile J and Chandler J, Mol Biol Cell 16, 757-768 (2005); both of which are incorporated by reference herein. However, the mechanisms driving the internalization and trafficking of BM proteins have not been explored.

The loss of apico-basal polarity is implicated in a number of diseases including polycystic kidney disease, retinitis pigmentosa, cystic fibrosis, interstitial cystitis, actinic keratosis, and a number of cancers, exemplified by bladder cancer (Wilson P D Biochimica et Biophysica Acta—Mol Basis Dis 1812, 1239-1248 (2011); Royer C and Lu X, Cell Death Diff 18, 1470-1477 (2011); both of which are incorporated by reference herein.) Methods that can be used to efficiently identify cells that have lost apico-basal polarity are clearly needed.

### SUMMARY

Methods of identifying cells that lack apico-basal polarity, methods of identifying test compounds that promote apico-basal polarity, methods of targeting payload molecules to diseased cells that lack apico-basal polarity, and compositions that facilitate these methods are disclosed herein.

Methods of rapidly identifying cells that lack apico-basal polarity involve contacting the cell with a reagent that binds dystroglycan or a homolog thereof. The reagent also comprises a label. The method further involves observing the assembly of the label on the cell surface or internalization of the label into acidic vesicles. Assembly of the label or internalization of the label into the cell is an indication that the cell lacks apico-basal polarity. The reagent that binds dystroglycan can be a protein such as laminin, perlecan, agrin, pikachurin, biglycan or any dystroglycan binding homolog or fragment thereof. The reagent can be a monoclonal antibody that binds dystroglycan or any fragment thereof. The label can be any label including a fluorescent label, radioactive isotope, or magnetic resonance imaging contrast agent. The cell can be any cell known to or suspected to lack apico-basal polarity including a cancer cell. The contacting of the cell with a reagent can be performed in vitro, ex vivo, or in vivo.

Methods of targeting a payload molecule to a cell that lacks apico-basal polarity involve contacting the cell with a protein that binds dystroglycan or a homolog thereof conjugated to a payload molecule. The payload molecule can be any agent that slows the growth of the cell (up to and including killing the cell) such as a radionuclide, a toxin, an siRNA, or a small molecule drug. The contacting of the cell with a reagent can be performed in vitro, ex vivo, or in vivo.

Pharmaceutical compositions disclosed herein include a dystroglycan binding protein and a payload covalently bound to the dystroglycan binding protein. One example of a dystroglycan binding protein is laminin, including fragments thereof. Examples of the payload include mertansine.

Methods of identifying test compounds that promote apico-basal polarity involve contacting a cell with the test compound, provided that the cell lacks apico-basal polarity. The methods further involve contacting the cell with a reagent that binds dystroglycan or a homolog thereof, provided that the reagent comprises a fluorescent label. A lack of assembly of the fluorescent label on the cell surface or a lack of internalization of the fluorescent label into acidic vesicles is an indication that the test compound promotes apico-basal polarity.

### BRIEF DESCRIPTION OF THE SEVERAL VIEWS OF THE DRAWINGS

Some of the Figures herein are better understood when provided in color. Applicants submit that color versions of such figures are part of the original disclosure and reserve the right to submit color versions of the figures herein in later proceedings.

FIG. 1 is a set of 12 images showing the results when E3D1 mammary epithelial cells (MEC)s were incubated for 18 hours with 10 μg/ml rhodamine-labeled laminin (Rhod-Ln) or unlabeled laminin. Unlabeled laminin was visualized using indirect immunofluorescence with anti-laminin antibodies followed by FITC-labeled secondary antibodies. Permeabilized cells were antibody labeled in the presence of 0.1% triton X-100, whereas non-permeabilized cells were antibody labeled without 0.1% triton X-100. Arrows indicate the presence of similar fibrillar assembled laminin. Note the lack of appreciable assembled endogenous laminin in the upper panels. All images were acquired with the same settings. Bar=25 μm. FIG. 2A is a still image of MECs after 10 min of incubation with Rhod-Ln. Time lapse images of the boxed region is shown in FIG. 2D. Bar=10 μm.

FIG. 2B is a set of time lapse images of MECs starting 10 min after addition of Rhod-Ln as in FIG. 2A and imaged every 5 min over a 50 min time period. Laminin was observed to coalesce into patches (white arrow). The bar is 5 μm.

FIG. 2C is a set of time lapse images of MECs starting 10 min after addition of Rhod-Ln as in FIG. 2A and imaged every 5 min over a 50 min time period. Laminin also formed long fibers (white arrow), similar to those seen in fixed images such as FIG. 1. The bar is 5 μm.

FIG. 2D is a set of time lapse images of MECs starting 10 min after addition of Rhod-Ln as in FIG. 2A and imaged every 5 min over a 50 min time period. Laminin was observed in relatively immobile patches that appeared to pinch off into vesicles and become highly mobile.

Arrow and arrowhead highlight the movement of two different mobile vesicles in each frame. The bar is 5 μm.

FIG. 2E is a plot showing the Steady-state dynamics of laminin internalization. E3D1 MECs were continuously incubated with Rhod-Ln and internalization quantified by flow cytometry after various times. Rhod-Ln continues to accumulate internally well after 24 hrs (n =3).

FIG. 3A is a set of three images showing MECs were incubated with CypHer-5 labeled laminin for 18 hrs at which point (t=0 seconds) live cells were imaged by time-lapse fluorescence microscopy over a 108 second time period. Imaging time is indicated in upper right of each panel. Dashed line in left panel outlines cell boundary. Live cell imaging of cypHer-Ln shows accumulation in acidic and mobile endocytic vesicles, most of which moved rapidly within the cytoplasm. Individual cyPher-Ln-filled vesicles are circled and color coded for tracking. The bar is 5 μm.

FIG. 3B is a set of three images showing MEC cells were treated with Rhod-Ln for 18 hrs, trypsinized to remove surface laminin, washed and fixed. The cells were labeled with conconavalin A (ConA) to reveal the cell plasma membrane. A single plane confocal scan clearly shows laminin filled vesicles within the cell (Merge). Bar=20 μm.

FIG. 4A is a line graph showing results where E3D1 MECs were pulse-labeled with

Rhod-Ln at 4° C. for 20 min, unbound Rhod-Ln washed away, and cells returned to 37° C. Samples were analyzed by flow cytometry at 0, 1, 2, 4, 8, 16, and 24 hrs post laminin labeling. (n=4).

FIG. 4B is a bar graph showing results where Pulse-labeled E3D1 MECs as in A were incubated in the absence (control) or presence of MG-132, leupeptin, or DMSO (vehicle) and analyzed by flow cytometry 24 hrs post laminin labeling. (n=4, * p<0.001).

FIG. 5A is a set of 9 images showing the results when: Following 18 hr incubation with Rhod-Ln (Ln), human breast epithelial UACC893 cells expressing Rab11-GFP shows no co localization of Ln with Rab11 expressing vesicles, whereas accumulation of laminin is clearly observed within Rab7-GFP and Lamp1-GFP expressing vesicles (arrows). n=nucleus. Bar=10 μm.

FIG. 5B is a set of three images showing the results of deconvolution imaging of Rhod-Ln (red) in cells expressing the GTPase-deficient mutant, RabS Q79L-GFP fusion (Rab5Q79L) (green). Accumulation of multiple individual laminin containing vesicles in multivesicular bodies of the late endosome is observed. Boxed XY image is shown at right. Vertical line indicates position of XZ scan shown at far right. Bar=5 μm.

FIG. 6A is a bar graph showing the results when E3D1 MECs were incubated with either 10 μg/ml Rhod-Ln or 40 μg/ml FITC-dextran (500S) in the presence or absence of DMSO (control), 100 μg/l heparin, 320 mM sucrose, or 40 μM dynasore for 18 hrs and processed for flow cytometry. All drug treatments resulted in significantly less laminin endocytosis (p<0.01), whereas no significant effect was observed with FITC-dextran (p>0.2, n=4).

FIG. 6B is a plot showing the results when E3D1 MECs were incubated continuously with Rhod-Ln or FITC-dextran. Internalization was quantified after 2, 4, 8, 16, and 24 hrs. Note that the rate of FITC-dextran internalization is more rapid and distinct from Rhod-Ln internalization.

FIG. 7A is a set of six images showing MEpG MECs which lack dystroglycan (DG) expression were either infected with empty vector (DG−/−) or WT DG (DG+), incubated with no laminin or Rhod laminin for 18 hours, trypsinized to remove surface laminin, washed and fixed.

Cells were labeled with conconavalin A (ConA) to reveal the cell plasma membrane. A single plane confocal scan shows abundant internal vesicles filled with laminin within DG expressing cells, but largely absent from DG−/− cells. Bar=20 μm.

FIG. 7B is a plot of laminin internalization in cells treated as in A was quantified by flow cytometry. The histogram demonstrates that DG expressing MECs (DG⁺) internalize significantly more laminin than DG lacking MECs (DG^{-/-}).

FIG. 7C is a bar graph of compiled mean fluorescence intensity flow cytometry data. DG+ MECs internalize 380% more laminin than DG^{-/-} MECs (*p<0.01, n=6).

FIG. 7D is a bar graph of E3D1cre19 MECs which lack β1 integrin (β1 Int) expression infected with either empty vector (β1 Int^{-/-}) or wild type β1 Int (β1 Int⁺). Laminin internalization was assayed by flow cytometry. No significant difference (N.S.) was found between β1 int-lacking cells (β1 int−/− vector) and WT β1 int (β1 Int+) expressing cells (p=0.17, n=6).

FIG. 8A is a set of three images showing MEC cells lacking DG (DG^{-/-}) were re-infected to express DG fused to GFP (dim green cells, DG⁺) or GFP alone (bright green cells, DG⁻). Rhod Ln was added to the culture and cells were imaged live at 37° C. Still image is taken from a 20 hr movie. Laminin (red) assembled only on the DG expressing dim green cells (arrows), and not on the bright green DG^{-/-} cells (arrowheads). Small laminin-positive vesicles are also seen only within dim green DG⁺ cells. These same vesicles can be seen rapidly moving within most of the DG+ cells. Bar=50 μm.

FIG. 8B is a set of four images showing cells co-expressing of DG-RFP (DG) and Rab7-GFP (Rab7) constructs treated with Alexa 647 labeled laminin (constructs treated with Alexa-Ln). Live cell imaging permitted the simultaneous tracking all three molecules, and revealed strong co-localization of DG and laminin within Rab7 vesicles of the late endosome. Arrows included for positional reference. Bar=10 μm.

FIG. 9A is a set of six images showing E3D1 cells were Rhod-Ln treated in the absence (Ln) or presence of the laminin fragments, E1′ (Ln+E1′) and E4 (Ln+E4) for 18 hrs. Both E1′ and E4 fragments prevented laminin assembly. Images were acquired under identical conditions.

FIG. 9B is a bar graph showing results of E3D1 cells incubated with Rhod-Ln for 18 hrs and internalized laminin was quantified by flow cytometry. No significant difference was observed between Ln fragment+Rhod-Ln treated cells and Rhod-Ln alone.

FIG. 9C is a bar graph showing results of E3D1 cells treated with Rhod-Ln labeled in the absence (vehicle) or presence of the MMP inhibitors GM6001 or marimistat for 18 hrs and internalized laminin quantified by flow cytometry. These MMP inhibitors show no significant effect on Rhod-Ln internalization.

FIG. 10A is an image of an immunoblot of MDA231 human breast carcinoma or human LN18 glioblastoma cells infected with empty vector (vector) or LARGE (glycosyltransferase-expressing) retrovirus. Probing with the glycosylation-specific anti-α-DG antibody IIH6 demonstrates the absence of glycosylated DG in vector infected cells and presence of glycosylated DG in LARGE infected cells. HA-tagged LARGE expression was detected using anti-HA antibodies, β-DG levels remain unchanged and demonstrates equal protein loading. Numbers on the left indicate locations of molecular weight markers (in kDa).

FIG. 10B is a set of eight images showing a laminin assembly assay. The assay shows that only LARGE expressing cells assemble laminin. Bar=50 μm. Insets of dashed boxed regions show detail of assembled laminin. Bar=5 μm.

FIG. 10C is a flow cytometry histogram of MDA-MB-231 cells infected with empty vector (black) or LARGE (red), incubated with rhod-Ln for 18 hours, and trypsinized for flow cytometry. A shift in fluorescence intensity to the right demonstrates much greater accumulation of laminin in LARGE-expressing cells compared to vector infected cells. The no laminin control histogram (No Ln) overlaps closely with the vector control.

FIG. 10D is a bar graph summarizing flow cytometry data as in (C) compiled from 3 separate experiments. MFI of MDA-MB-231 vec=0.1+/−0.014, LARGE=6.82+/−0.241, p<0.001; LN18 vec=0.145±0.155, LARGE=1.49±0.049, p<0.05, n=3.

FIG. 10E is a set of six images showing MDA-MB-231 cells were transfected with the Rab7-GFP fusion protein (green) and incubated with Rhod-Ln for 18 hrs. Fluorescence imaging of Rhod-Ln (red) in cells without (control) and with expression of LARGE (LARGE) demonstrates strong accumulation of laminin in Rab7 expressing vesicles only in LARGE expressing cells (arrows in merged image). Bar=10 μm.

FIG. 11A is an image of an immunoblot showing loss of DG and β1 integrin expression in respective cell lines. 20 μg/lane of protein extract from the indicated cell lines were resolved by SDS-PAGE, immunoblotted with antibodies to the proteins indicated at the right. Actin and E-cadherin were used as a protein loading control and epithelial cell marker, respectively. The dashed line indicates respectively that two columns were removed that contained extract from cell lines not described in this paper. Numbers on the left indicate locations of molecular weight markers (in kDa).

FIG. 11B is a line graph showing E3D1cre19 MECs pulse treated with Rhod-Ln as described above. The MFI of internalized Ln was quantified by flow cytometry to reveal the rate of laminin internalization in β1 integrin-expressing (B1 Int+) and knockout (B1 Int−/−) cells (n=3).

FIG. 11C is a bar graph showing MECs were labeled with laminin and trypsinized as described above. Cellular fluorescence intensity of Rhod-Ln was quantified by flow cytometry, background subtracted, the means compiled from three separate experiments, and normalized to Ln alone control. Blocking Ln binding to β1 integrin with the Hat2/5 antibody produced a 36% decrease in Ln internalization p<0.05, n=3.

FIG. 12 is a set of eight images showing the killing of carcinoma cell killing by a laminin-DM1 bioconjugate. Purified murine laminin-111 was conjugated to the cytoxin DM1 using a SMCC linkage. The bladder carcinoma cell line UMUC5 was treated with 10 nM laminin-DM1 (L-DM1) or with the vehicle control (phosphate-buffered saline) in the presence of CellEvent™ (Life Technologies), a fluorescent cell death indicator (green). Phase and fluorescent imaging shows complete cell killing 48 hours post L-DM1 exposure, demonstrating that the L-DM1 conjugate can deliver and release a cytotoxin to the carcinoma cell interior.

FIG. 13A is an image of the mammary epithelial line E3D1 grown at high density to form a polarized monolayer, with apico-basal polarity shown by tight junction formation (ZO-1 immmunostaining, red) that appears apical to the cell nuclei (dapi staining, blue). The monolayer was then mechanically disrupted in selected regions by scratching with a pipette tip. Laminin binding and internalization was subsequently assayed by fluorescence microscopy 20 hours after the addition of rhodamine-labeled laminin to the culture medium.

FIG. 13B is an image showing that laminin binding and internalization (red) was strongly suppressed within the polarized cell monolayer (left side), but evident in cells migrating from the leading edge of the disrupted region (arrows). Dapi staining (blue) shows cell localization. BRDU detection shows dividing cells (green).

FIG. 13C is an image in higher magnification than 14B showing that laminin binding (red) occurs on cells lacking a contiguous ring of adherens junctions.

FIG. 13D is an image (arrows in D) as detected by β-catenin immunostaining (green) showing the adherens junctions of the cells in 14C.

FIG. 13E is an image showing dapi staining of the nuclei shown in cells 14C and 14D. FIG. 13F is a merged image of the images of FIGS. 14C-14E. Laminin binding and internalization are absent in cells where the adherens junctions form a contiguous ring, and apico-basal polarity is maintained. (bars=30 μm in FIGS. 13B-13F).

### SEQUENCE LISTING

SEQ ID NO: 1 is a protein sequence of human dystroglycan

SEQ ID NO: 2 is a protein sequence of a human laminin alpha 1 precursor

SEQ ID NO: 3 is a protein sequence of a human laminin-211 LG4-5 domain.

SEQ ID NO: 4 is the protein sequence of a mouse laminin-111 LG4-5 domain.

### DETAILED DESCRIPTION

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632 02182-9); and Robert A. Meyers (ed.), Molecular Biology and Biotechnology: a Comprehensive Desk Reference, published by VCR Publishers, Inc., 1995 (ISBN 1-56081-569-8). Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” In addition, the materials, methods, and examples are illustrative only and not intended to be limiting. In order to facilitate review of the various embodiments of the disclosure, the following explanations of specific terms are provided:

Antibody: A polypeptide including at least a light chain or heavy chain immunoglobulin variable region which specifically recognizes and binds an epitope of an antigen (such as dystroglycan) or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W. H. Freeman & Co., New York, 1997.

Apico-basal polarity: the differential expression of proteins and other structures between an apical or “top” side of a cell and a basal or “bottom” side of a cell. In an epithelial cell such as a bladder epithelial cell, the apical side is the side facing the lumen (for example, the lumen of an intestine or the bladder) and the basal side is the side away from the lumen. This polarity is evident in many aspects of epithelial cell architecture, including the polarized distribution of organelles within the cells (e.g. nucleus and Golgi apparatus), the polarized orientation of cell surface proteins and adhesive junctions, and the directional regulation of protein trafficking in accordance with the apical and basal domains. Other cell types can also display apico-basal polarity. For example, a leukocyte can adopt apico-basal polarity when migrating to the source of a chemokine, when it binds to a vessel wall, or during the process of extravasation. A lack of apico-basal polarity (also known as apical-basal polarity) is implicated in a number of disease states including polycystic kidney disease, retinitis pigmentosa, cystic fibrosis, interstitial cystitis of the bladder, and a number of cancers. Apico-basal polarity can be identified by any of a number of methods including the detection of the presence of apically polarized of tight junctions between cells and the polar distribution of cellular organelles and cell surface proteins.

Binding or stable binding: An association between two substances or molecules, such as the association of a molecule of dystroglycan with another other biological macromolecule such as a laminin or other dystroglycan binding molecule. Binding can be detected by any procedure known to one skilled in the art, such as by physical or functional properties. Binding can also be detected by visualization of a label (such as a fluorescent label) conjugated to one of the molecules.

Cancer: A disease or condition in which abnormal cells divide without control and are able to invade other tissues. Cancer cells spread to other body parts through the blood and lymphatic systems. Cancer is a term for many diseases. There are more than 100 different types of cancer in humans. Most cancers are named after the organ in which they originate. For instance, a cancer that begins in the bladder may be called a bladder cancer. However, the characteristics of a cancer, especially with regard to the sensitivity of the cancer to therapeutic compounds, are not limited to the organ in which the cancer originates. A cancer cell is any cell derived from any cancer, whether in vitro or in vivo.

Cancer is a malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

“Metastatic disease” or “metastasis” refers to cancer cells that have left the original tumor site and migrate to other parts of the body for example via the bloodstream or lymph system. The “pathology” of cancer includes all phenomena that compromise the wellbeing of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

Most carcinomas (cancers of epithelial origin) are characterized by the loss of apico-basal polarity that arises during cancer progression. Such carcinomas can include lung cancers, breast cancers, skin cancers (such as actinic keratosis which leads to squamous cell carcinomas) bladder cancers, and colon cancers, among others (Liu Y & Chen L P, J Cancer Res Ther Suppl 2, S80-585 (2013); Hinck L & Nathke I, Curr Opin Cell Biol 26, 87-95 (2014); and Nese N et al, J Natl Compr Canc Netw 7, 48-67 (2009); all of which are incorporated by reference herein).

Contacting: Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Control: A reference standard. A control can be a cell that is known to have lost apico-basal polarity and is known to aggregate and/or internalize dystroglycan at a particular rate (positive control). A control can also be a cell known not to have lost apico-basal polarity and therefore does not aggregate or internalize dystroglycan.

Domain: any part of a polypeptide that can be demonstrated to mediate a particular protein function.

Effective amount: An amount of agent, such as a pharmaceutical composition comprising a molecule that specifically binds dystroglycan conjugated to a payload molecule that is sufficient to generate a desired response, such as slowing the growth of a cancer cell. In some examples, an “effective amount” is one that treats (including prophylaxis) one or more symptoms and/or underlying causes of any of a disorder or disease. An effective amount can be a therapeutically effective amount, including an amount that prevents one or more signs or symptoms of a particular disease or condition from developing. Label: A detectable compound or composition that is conjugated directly or indirectly to another molecule to facilitate detection of that molecule. Specific, non-limiting examples of labels include fluorescent tags, enzymes, radioactive isotopes, molecules that specifically bind other molecules (e.g. biotin or streptavidin) and compounds visible in MRI imaging such as MRI contrast agents. In some examples, a label is attached to a reagent that binds dystroglycan, such as a laminin or fragment thereof or antibody that binds dystroglycan.

Polypeptide: Any chain of amino acids, regardless of length or posttranslational modification (such as glycosylation, methylation, ubiquitination, phosphorylation, or the like). “Polypeptide” is used interchangeably with “protein,” and is used to refer to a polymer of amino acid residues. A “residue” refers to an amino acid or amino acid mimetic incorporated in a polypeptide by an amide bond or amide bond mimetic.

Subject: A living multicellular vertebrate organism, a category that includes, for example, mammals and birds. A “mammal” includes both human and non-human mammals, such as mice. In some examples, a subject is a human patient having or suspected of having a disease characterized at least in part by the loss of apico-basal polarity.

Sequence identity/similarity: Sequence identity/similarity/homology: The identity/homology between two nucleic acid sequences, or two amino acid sequences, is expressed in terms of the similarity between the sequences, otherwise referred to as sequence identity. Sequence identity is frequently measured in terms of percentage identity (or homology, the terms are interchangable); the higher the percentage, the more homologous the two sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBl Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein. When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBl web site.

One of skill in the art will appreciate that the particular sequence identity ranges are provided for guidance only; it is possible that strongly significant homologs could be obtained that fall outside the ranges provided particularly if those homologs have a similar or identical function and a similar or identical level of activity to one another.

Identification of Cells that have Lost Apico-Basal Polarity

### Methods of Identifying a Cell as Lacking Apico-Basal Polarity

Epithelial cells are a basic cell type of animals that line the internal or external surfaces of many organs, and have specialized functions in the directional secretion or absorption of molecules to and from tissue cavities, and in the protection of underlying cell layers from the external environment. In accordance with their functions, these cell are inherently oriented or “polarized”, have a distinct “top” and “bottom” referred to as the apical and basal (or baso-lateral) domains. The apical domain faces the external environment or lumen of cavities, whereas the basal domain faces the internal tissues and blood supply. This polarity is referred to apico-basal polarity. This polarity is evident in many aspects of epithelial cell architecture, including the polarized distribution of organelles within the cells (e.g. the nucleus and Golgi apparatus), the polarized orientation of cell surface proteins and adhesive junctions, and the directional regulation of protein trafficking in accordance with the apical and basal domains. A hallmark of this apico-basal polarity is the separation of the cell's plasma membrane into apical and basal domains, and the segregation of cell-surface proteins between these domains. This molecular segregation is enabled by the formation and maintenance of the cell-cell junctions, comprised of the adherens and tight junctions, which form a physical barrier to the diffusion of membrane proteins within the lipid bi-layer. With this barrier intact, proteins directed uniquely to the basal-lateral membrane domain are restricted from the apical domain, and vice versa. The loss of apico-basal polarity is implicated in a number of diseases including polycystic kidney disease, retinitis pigmentosa, cystic fibrosis, interstitial cystitis and carcinomas (Wilson P D Biochimica et Biophysica Acta—Mol Basis Dis 1812, 1239-1248 (2011); Royer C and Lu X, Cell Death Diff 18, 1470-1477 (2011); both of which are incorporated by reference herein.) Loss of apico-basal polarity is a hallmark of disease, and possibly a driving force in disease progression. The adherens and tight junctions are targets of congenic protein signaling, and loss of integrity in these junctions is an early event in cancers (Khursheed, M. & Bashyam, M. D, J Biosci 39, 145-155 (2014); incorporated by reference herein).

Methods that can be used to efficiently identify cells that have lost apico-basal polarity are clearly needed because they can be used for the detection of diseased cells and also for the targeted treatment of diseased cells. The loss of polarity is most often detected by analysis of tissue biopsies, using fixed and stained tissue slices, looking at the orientation of cell nuclei, Golgi and other markers such as the polarized secretion of extracellular molecules. (Malon C et al, U.S. Pat. No. 8,655,035 (2014); incorporated by reference herein). However, methods to detect the loss of polarity in living, intact tissues are lacking.

One opportunity to detect the loss of polarity in living tissues is through sensing the redistribution of cell surface proteins that occurs with breakdown of the cell-cell junctions that establish the apico-basal membrane barrier. For example, the mixing or mislocalization of typically apical or baso-lateral proteins at the cell surface would indicate the loss of polarity. Detection of this mislocalization can be achieved using affinity agents binding to domain-specific membrane molecules. For example, this can be achieved through the exposure of the apical cell surface to an affinity agent (e.g. ligand or antibody) that binds to a typically baso-lateral cell surface molecule. In this scenario, the absence of binding at the apical cell surface would indicate the maintenance of apico-basal polarity (i.e. intact segregation of the apical and basal membrane domains), and the presence of binding would indicate the loss of polarity (i.e. a breakdown in the barrier between the two membrane domains). The coupling of an imaging or contrast agent to the cell-binding agent would enable detection of binding by a variety of methods. The cell binding agent, conjugated to an imaging or contrast agent, would therefore comprise a molecular sensor for the loss of polarity.

Measurement of sensor binding to the apical cell surface can be achieved by detecting the binding at the cell surface and also by detecting the internalization of the sensor into the cell interior. Membrane proteins on the cell surface, and the ligands that bind them, can be internalized through varied mechanisms of endocytosis. Endocytic internalization of cell surface proteins and their ligands occurs at different rates and efficiencies, and pass through different endocytic pathways (Duncan R & Richardson S C, Mol Pharm 9, 2380-2402, (2012); incorporated by reference herein). Importantly for measures of sensor binding, the abundance of the surface protein, the efficiency of endocytosis, the kinetics of endocytosis and the pathways of endocytosis can each be either advantageous or disadvantageous to signal detection. For example, a high rate of internalization and a long duration of retention within the cell could, in many cases, enhance a detection signal. Conversely, a low rate of internalization and/or a rapid degradation or recycling of the signal (either by chemical degradation or release from the cell) could reduce the detection signal.

The endocytic internalization of such a sensor offers the important added advantage that cell binding agents including any linked payload molecules, can be selectively delivered to the cell interior for treatment of the diseased cell. For example, a cytotoxin can be delivered to the cell interior to kill the diseased cell (e.g. for cancer treatment) or a therapeutic can be delivered to the cell interior for the correction of a cellular defect, such as siRNA or kinase inhibitor. In this scenario, a cell exhibiting intact apico-basal polarity will be unable (or resistant) to internalizing the therapeutic from the apical domain when targeting a typically baso-lateral cell surface molecule, and vice versa. Upon loss of apico-basal polarity, this resistance would disappear, and selective targeting of the diseased cell would result.

Disclosed herein are compounds and methods of identifying and targeting cells that have lost apico-basal polarity that involve the use of compositions that specifically bind the cell surface glycoprotein dystroglycan. Dystroglycan is a prominent and widely expressed cell surface protein. Dystroglycan is a highly efficient mediator of endocytosis in a wide range of cell types, being more effective at internalization that related molecules such as the β1 integrins (Leonoudakis D et al, J Cell Sci 127, 4894-4903 (2014); incorporated by reference herein). Dystroglycan is restricted from the apical membrane domain of polarized epithelial cells, and a labeled dystroglycan binding molecule can detect the absence or loss of apico-basal polarity when introduced from the apical surface. The kinetics of internalization are very slow, showing that molecules internalized by dystroglycan have a long duration in the cell interior, allowing for a durable detection signal. We have found that dystroglycan traffics bound molecules to the lysosome, which is advantageous for the activation of certain drugs or drug conjugates. Therefore, dystroglycan-binding compositions can be used to selectively and efficiently target imaging agents and therapeutic agents to cells lacking apico-basal polarity.

Methods of identifying a cell as lacking apico-basal polarity involve contacting the cell with a reagent that binds dystroglycan or any functional mutant, homolog, or ortholog thereof. The reagent can bind human dystroglycan, mouse dystroglycan, or any other mammalian homolog of dystroglycan, or any mutant thereof that can still be (a) recognized by the reagent as dystroglycan or (b) shown to be a functional dystroglycan molecule using techniques such as those described in the Examples below. Examples of the reagent include recombinantly produced ligands of dystroglycan such as laminin, perlecan, agrin, pikachurin, biglycan or any other such ligand or any fragment of any such ligand that binds dystroglycan such as the mouse laminin-111 LG45 domain and the human laminin-211 LG4-5 domain (Harrison D et al, J Biol Chem 282, 11573-11581 (2007); incorporated by reference herein). The reagent can further comprise a monoclonal antibody or any antigen binding fragment thereof that binds dystroglycan.

The reagent further comprises a label. The label can be conjugated to the dystroglycan binding molecule. The label can be any fluorescent, enzymatic, magnetic, metallic, chemical, or other label that signifies and/or locates the presence of specifically bound reagent. The label can be a label that can be detected on the cell surface and/or intracellularly, such as a fluorescent label that can be detected by flow cytometry. In other examples, the label can be detected through the use of magnetic resonance imaging (MRI), also known as an MRI contrast agent. An MRI contrast agent is a reagent used to improve imaging of internal body structures. Some MRI contrast agents comprise gadolinium (Gd). Other MRI contrast agents can comprise iron oxide, iron platinum, and manganese, among others. In some examples the MRI contrast agent is incorporated into a chelate, which is in turn conjugated to the dystroglycan binding reagent.

The concept of a dystroglycan binding reagent also incorporates systems in which the dystroglycan binding moiety and the label are included in separate polypeptides. For example, the dystroglycan binding reagent can be bound to the cell and then a second reagent that binds the dystroglycan binding reagent can be contacted with the cell. For example, if the dystroglycan binding agent comprises laminin, then a labeled anti-laminin antibody can be bound to the laminin, thereby labeling the dystroglycan.

The method further involves observing assembly of the label on the surface of a cell or observing internalization of the label into acidic vesicles. The method can further involve observing both the assembly of the label on the surface of the cell and the internalization of the label into acidic vesicles. The techniques used in observing the assembly of the label on the surface of the cell and/or observing the internalization of the label in the acidic vessels will depend on the type of label used and whether or not the observation is of the assembly, the internalization, or both. For example, a fluorescent label can be observed assembling on the surface of the cell by fluorescence microscopy. Internalization of a fluorescent label can be observed using flow cytometry. Assembly or internalization of an MRI contrast agent can be observed using magnetic resonance imaging. One of skill in the art would be able to select the type of label appropriate for the type of detection used.

The identification of a cell that lacks apico-basal polarity through observing assembly/internalization of a labeled dystroglycan binding protein can be used for any of a number of downstream purposes. For example, identification of a cell that has lost apico-basal polarity using a dystroglycan binding protein labeled with an MRI contrast agent can indicate recurrence of bladder cancer. Alternatively, identification of a cell that has lost apico-basal polarity using a fluorescent label can signal cancerous tissue that can further be removed using fluorescence-guided surgery (Pan Y et al, Sci Transl Med 6, 260ra148 (2014); incorporated by reference herein).

Methods of Identifying Test Compounds that Restore Apico-Bbasal Polarity

Methods of identifying test compounds that restore apico-basal polarity involve adding a test compound to a cell that lacks apico-basal polarity and also adding to the cell the labelled reagent that specifically binds dystroglycan described above. Assembly of the label on the cell surface and/or internalization of the label into acidic vesicles can be observed as described above. Test compounds that prevent assembly of the label on the surface and internalization of the label into acidic vesicles are identified as compounds that restore apico-basal polarity.

A test compound can be any small molecule, natural product, protein, aptamer, siRNA, or any other molecule that could be used to contact a cell. A test compound is generally provided in a vehicle, such as a solvent. The vehicle can be any appropriate solvent including compositions comprising water, ions, or organic compounds. Examples of vehicles include buffered saline or other buffered solvents or DMSO or other organic solvents. A test compound can also be a compound known to restore apico-basal polarity that can be used as a positive control. A test compound can also be a compound known not to restore apico-basal activity that is used as a negative control (or the vehicle alone can be used). The methods herein can be used to screen a plurality of test compounds, also described as a library of test compounds. The methods herein can be further adapted to high throughput screening of a set of test compounds in batches of 96, 384, or 1048 on assay plates adapted for such screening.

### Compositions and Methods Used in Targeting a Payload Molecule to a Cancer Cell

A dystroglycan binding molecule can be conjugated to a payload molecule. In general, the payload molecule is a molecule that is detrimental to the growth or further survival of the cell to which the reagent binds. The payload molecule can comprise a small molecule drug, a protein, an siRNA, a nanoparticle, a radionuclide (including a chelated radionuclide), a subunit of a pore forming complex, or any other payload molecule that can be conjugated to the dystroglycan binding molecule and result in the slowing of growth (up to and including stopping growth) of the cell to which the reagent binds. Said toxicity would be selective due to the assembly of the dystroglycan binding molecule/payload complex on the apical surface and/or the internalization of the dystroglycan binding molecule payload complex into the cell. In some examples, the payload molecule comprises mertansine, also known as DM1. Mertanisine has the structure shown below.

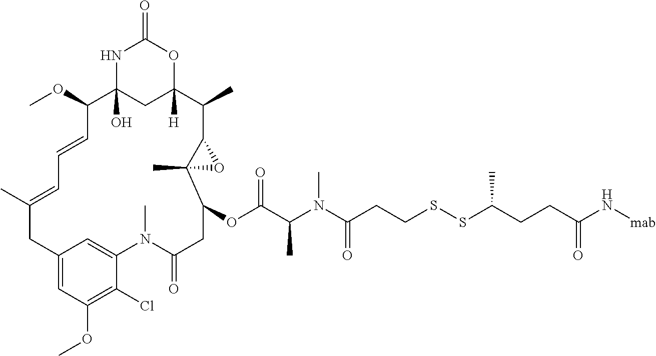

Targeting can occur in vitro, ex vivo, or in vivo.

### Methods and Compositions Useful in Detecting a Bladder Abnormality

Disclosed herein are methods of detecting a bladder abnormality in a subject. In particular, the examples include contacting a bladder cell with a reagent that binds dystroglycan. The reagent further comprises a label. Assembly of the reagent on the surface of the cell or internalization of the reagent into acidic vesicles of the bladder cell (either of which is observed through detection of the label) indicates the presence of a bladder abnormality. The reagent can be any reagent that binds dystroglycan including a labeled ligand of dystroglycan or any dystroglycan binding domain thereof. Other examples of the reagent can be a labeled dystroglycan binding antibody.

One example of a reagent that binds dystroglycan is a laminin. Laminins are major signaling and structural molecules of BMs and modulate a host of cellular functions, including cell polarity, survival, and hormone signaling (Domogatskaya et al, 2012 supra; Hohenester E and Yurchenco P D, Cell Adh Migr 7, 56-63 (2013) incorporated by reference herein; Leonoudakis D et al, J Cell Sci 123, 3683-3692 (2010) incorporated by reference herein; Streuli CH et al, J Cell Biol 129, 591-603 (1995) incorporated by reference herein; Yurchenco and Patton, 2009 supra). Laminins were reported over two decades ago to be internalized by cells but the mechanisms involved remain uninvestigated (Coopman P et al, Eur J Cell Biol 56, 251-259 (1991); Liotta L A et al, Anticancer Drug Des 2, 195-202 (1987); both of which are incorporated by reference herein). Consequently, little is known about the pathways and mechanisms controlling the endocytic trafficking of laminins or other BM proteins. Herein, the laminin receptor, dystroglycan (DG) is identified as the dominant regulator of laminin endocytosis. DG is known to be functionally compromised in many cancers (Akhavan et al, 2012 supra), suggesting laminin internalization defects. Indeed, restoration of DG function dramatically enhanced the internalization and trafficking of laminin in breast cancer and glioblastoma cells. Results presented here uncover novel mechanisms regulating normal cell-BM interactions and identify these mechanisms as compromised in a broad range of cancers.

Bladder abnormalities detectable by this invention include bladder cancer and interstitial cystitis.

### EXAMPLES

### Example 1—Laminin is Rapidly Internalized in Functionally Normal Cells

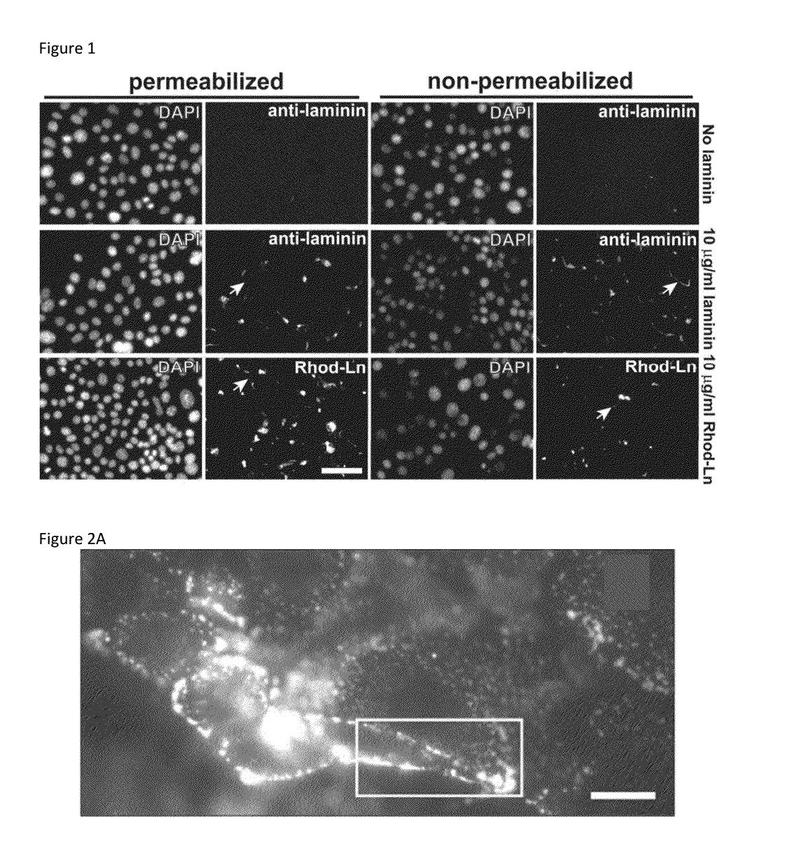

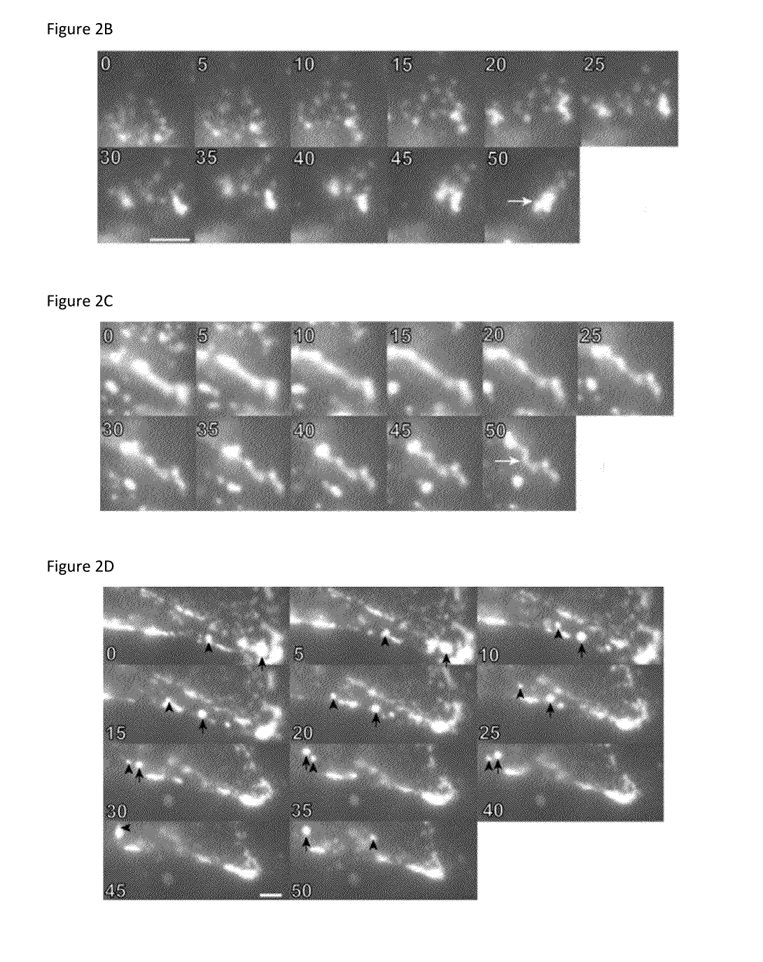

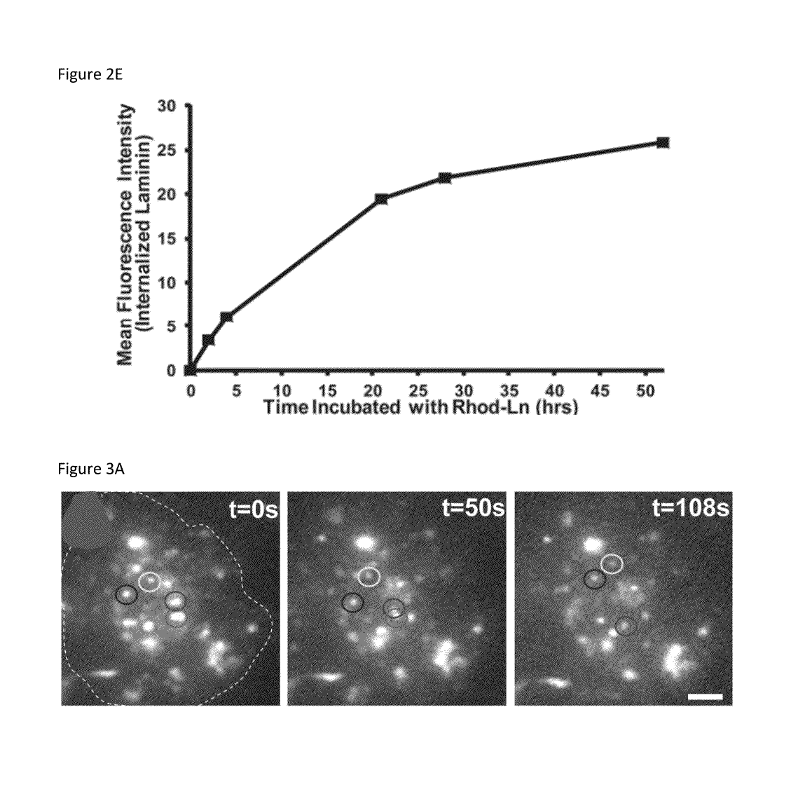

Direct labeling of laminin-111 (hereafter called laminin) has been previously used to assay the mechanisms of receptor-facilitated laminin assembly on the surface of living cells (Akhavan A et al, 2012 supra; Leonoudakis et al, 2010 supra; Weir M L et al, J Cell Sci 119, 4047-4058 (2006); incorporated by reference herein). As observed previously, fluorescently-labeled laminin assembled on the surface of functionally normal mammary epithelial cells (MECs) in the same manner as unlabeled laminin (FIG. 1) (Leonoudakis et al, 2010 supra; Weir Let al, 2006 supra). Endogenous laminin production was barely detectable in these cells, and did not contribute significantly to the assembled laminin in the described assays using exogenous laminin (FIG. 1). Time-lapse imaging revealed binding of rhodamine-labeled laminin (Rhod-Ln) to the surface of mammary epithelial cells (MECs) which coalesced into small patches within 10 minutes (FIG. 2A). Over a 50 min time period, laminin patches were found to form into larger clusters and fibrils (FIGS. 2B and 2C, arrows) resembling laminin assemblies observed after 18 hours of incubation. Unexpectedly, the budding of laminin laden vesicles internally from the cell surface was observed. These vesicles moved throughout the cytoplasm within 10 minutes of exposure to Rhod-Ln (FIG. 2D, black arrowheads).

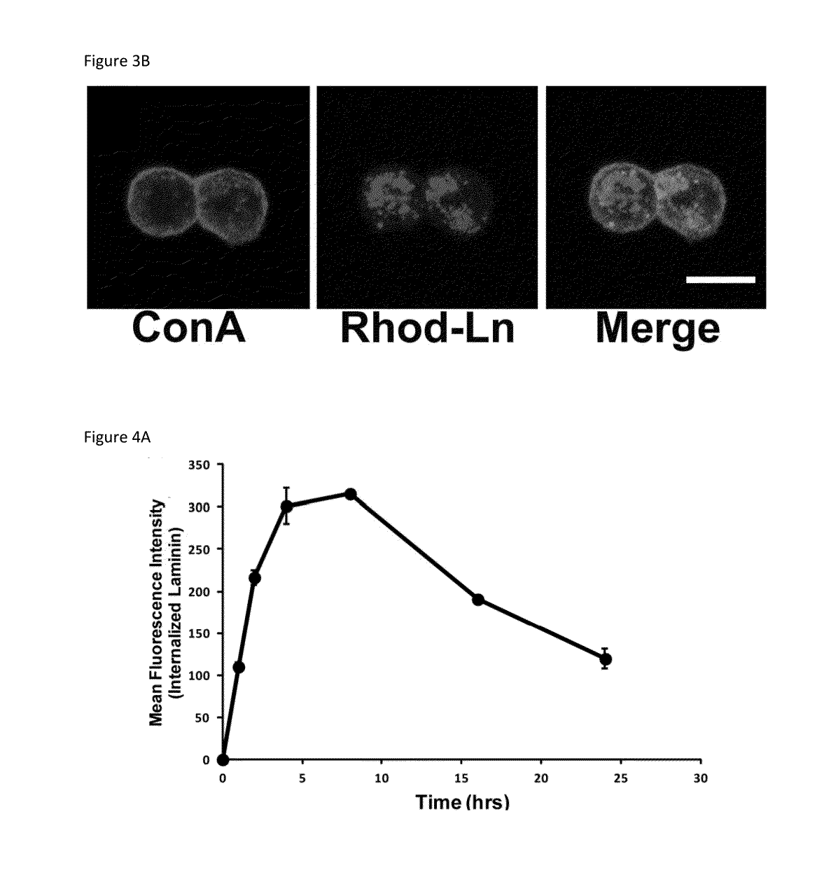

Endocytic internalization of laminin was confirmed by multiple methods. Laminin was labeled with the pH-sensitive fluorescent label CypHer-5 (CyPher-Ln) to exclusively image internalized laminin in attached, living cells. The fluorophore CypHer-5 is non fluorescent at pH 7.4 and maximally fluorescent at pH 5.5, permitting fluorescence detection of laminin within intracellular acidic vesicles (pH 4.8-6.0). Following overnight incubation of MECs with CypHer-Ln, live cell imaging detected bright fluorescent vesicles moving rapidly within the cytoplasm (FIG. 3A). Intracellular laminin within the cytoplasm was also independently observed via removal of surface bound laminin followed by confocal imaging. MECs were exposed to 10 μg/ml Rhod-Ln for 18 hrs, trypsinized, washed with PBS/EDTA to remove surface-bound laminin, allowed to re-attach, and stained with the membrane marker FITC-concanavalin A (conA). Confocal imaging revealed undetectable surface laminin (no overlap with plasma membrane conA) and abundant laminin in internalized vesicles (FIG. 3B). This method of cell treatment permitted a quantitative, flow cytometry-based assay of laminin internalization. In this assay, cells incubated with Rhod-Ln were trypsinized, washed (as in FIG. 3B), and the remaining internal Rhod-Ln fluorescence quantified by flow cytometry. Using this assay, laminin internalization was confirmed in diverse cell types including in primary mammary epithelial cultures, mammary epithelial cell lines (E3D1, MEpG), human fibroblasts (NIH 3T3 cells), primary astrocytes, and human cancer cell lines (breast and glioma).

### Example 2—The Dynamics of Laminin Internalization Point to Lysosomal Degradation

To explore the dynamics of laminin internalization, steady-state time course experiments (measuring internalization in the continuous presence of labeled laminin) and pulsed time course experiments were performed. In pulsed time course experiments, the cells were exposed to exogenous Rhod-Ln at 4° C. to prevent internalization, excess unbound Rhod-Ln was washed away, and then cells were returned to 37° C. to allow synchronized internalization and trafficking to proceed. Steady-state laminin internalization assays revealed that internalized laminin was measurable within 1 hour and did not plateau until after 30 hours (FIG. 2E), reflecting the continuous uptake of exogenous soluble laminin. Following pulsed and synchronized laminin exposure, Rhod-Ln was again observed to internalize within 1 hour, but reached a maximum at 8 hours, after which the levels of internal laminin declined (FIG. 4A). After 24 hours, internal Rhod-Ln levels declined to ˜37% of the maxima, indicating degradation (MFI: 315±3.5 to 119±11.5). To determine if laminin was degraded using classical degradation pathways, pulsed time course experiments in the presence of leupeptin (lysosome inhibitor) or MG-231 (proteasome inhibitor) were performed. In the presence of leupeptin, a 153% increase of (105.5±3 vs. 266±11.2; n=4; p<0.001) in internal Rhod-Ln compared to vehicle controls was observed (FIG. 4B). Inhibition of the proteasome with MG-231 also increased internal Rhod-Ln by 59% relative to vehicle controls (105.5±3 vs. 167.5±7; n=4; p<0.001). These data indicate that laminin internalized by epithelial cells is degraded primarily by lysosomes, and degradation is detectable at more than 8 hours post internalization.

### Example 3—Laminin is Trafficked through Multivesicular Bodies of the Late Endosome to the Lysosome

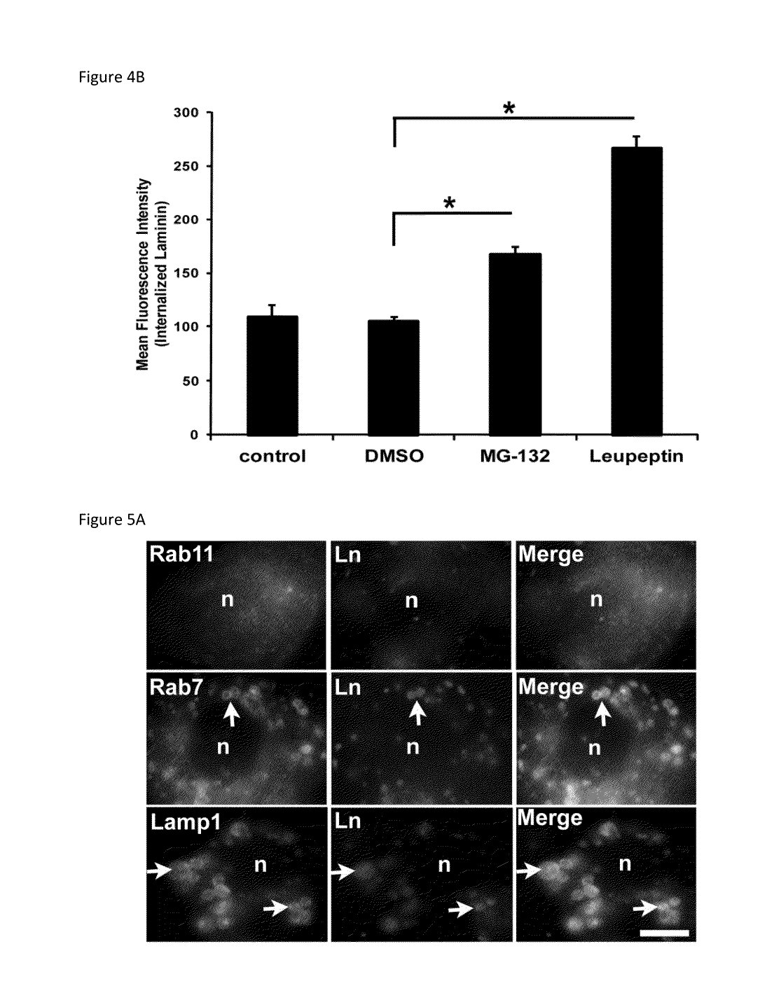

The pathway of laminin internalization was tracked using live cell imaging and transient expression of Rab-GFP fusion proteins to label distinct vesicles. Strong co-localization of internalized laminin was observed in conjunction with the Rab7 marker for late endosomes (FIG. 5A, middle panels) and within lysosomes labeled with the lysosomal associated membrane protein 1 (Lamp1)-GFP fusion protein (FIG. 5A, lower panels). No significant co-localization was observed with Rab11-containing vesicles of the recycling endosome at time points up to 18 hours (FIG. 5A, upper panels). The relatively slow movement of the laminin-laden vesicles matched the movement of Rab7 and lysosomal markers and was clearly distinct from the rapid movement of Rab11 vesicles. Additionally, laminin was clearly observed within multivesicular bodies of the late endosome, particularly when these bodies are enhanced by expression of a GTPase deficient mutant of Rab5 (Rab5Q79L-GFP), causing the fusion of early and late endosomes (Duclos S et al, J Cell Sci 116, 907-918 (2003); incorporated by reference herein) (FIG. 5B). These data reveal that internalized laminin traffics predominantly to the late endosome and lysosome.

### Example 4—Laminin Internalization is Receptor-Mediated

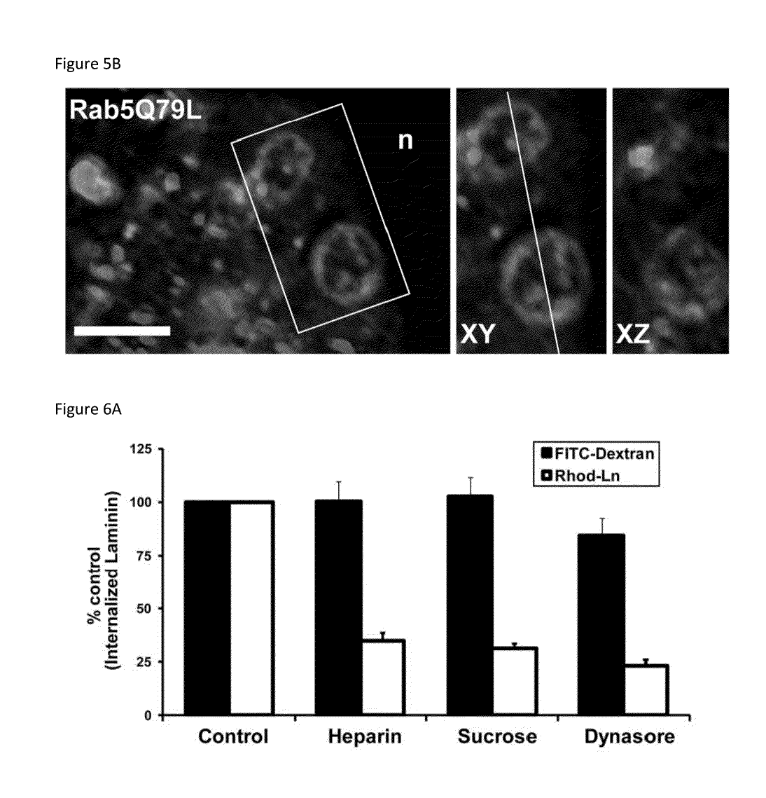

Laminin internalization could be mediated by either receptor-dependent or receptor independent mechanisms (e.g. pinocytosis). Steady-state laminin internalization was measured using flow cytometry in the presence of potential inhibitors and compared to internalization of 500S FITC-dextran, a molecule of similar molecular size to laminin known to be endocytosed by receptor-independent mechanisms. The specific inhibitor of dynamin, dynasore (Macia E et al, Dev Cell, 839-850 (2006); incorporated by reference herein), inhibited internalization of laminin by >75% (FIG. 6A). Under hypertonic sucrose, a condition known to inhibit receptor-mediated endocytosis (Heuser J E and Anderson R G, J Cell Biol 108, 389-400 (1989); incorporated by reference herein) laminin internalization was reduced by 65% (FIG. 6A). Addition of heparin, a molecule known to bind the laminin LG4-5 domain (Harrison D et al, J Biol Chem 282, 11573-11581 (2007); incorporated by reference herein), decreased laminin internalization by 69% relative to controls (FIG. 6A). In contrast, internalization of FITC-dextran was not significantly changed by any of these reagents (FIG. 6A). Additionally, a 24 hour time course of Rhod-Ln and FITC-dextran internalization revealed clearly different internalization dynamics (FIG. 6B). Specifically, under steady state, laminin internalization was nearly linear throughout the 24 hour time course, whereas dextran internalization plateaued after 16 hrs. Combined, these results indicate that laminin internalization is receptor-mediated and regulated by the GTPase dynamin.

### Example 5—Dystroglycan is the Predominant Mediator of Laminin Internalization

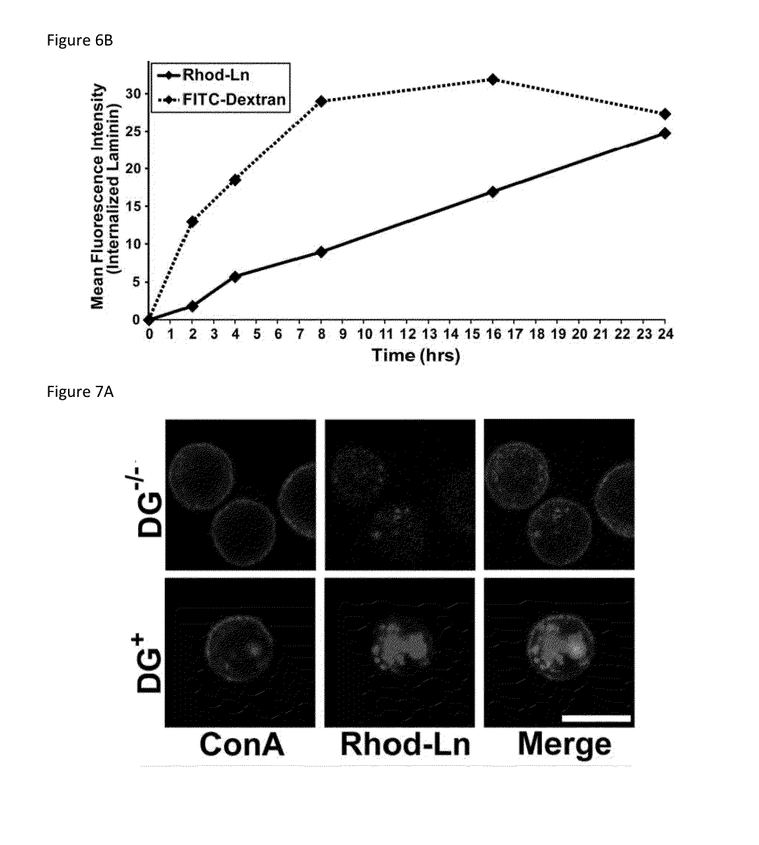

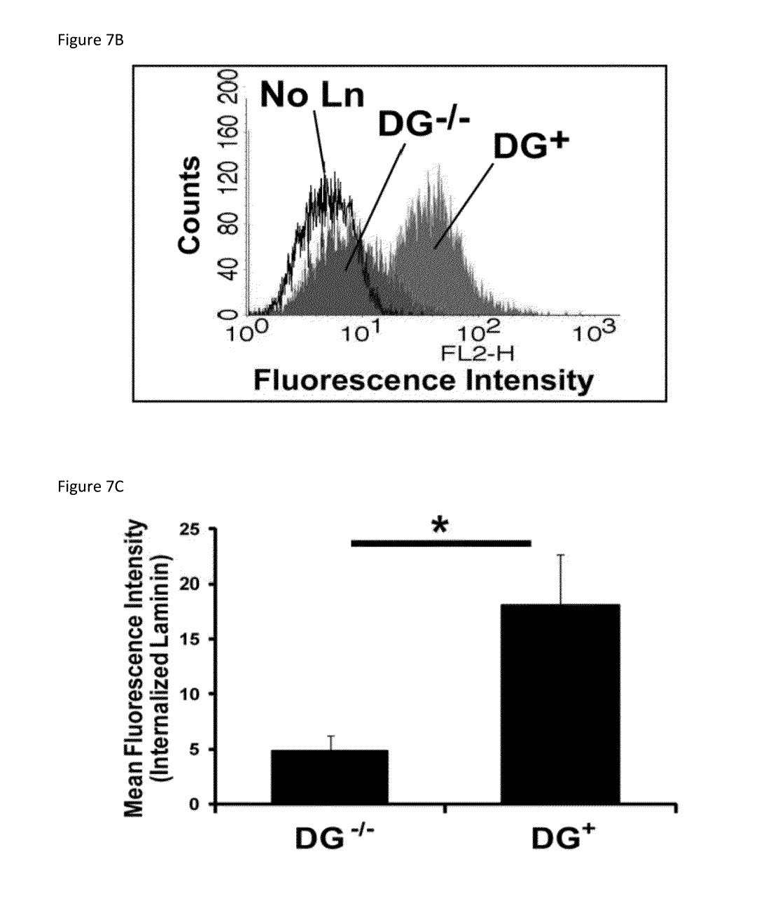

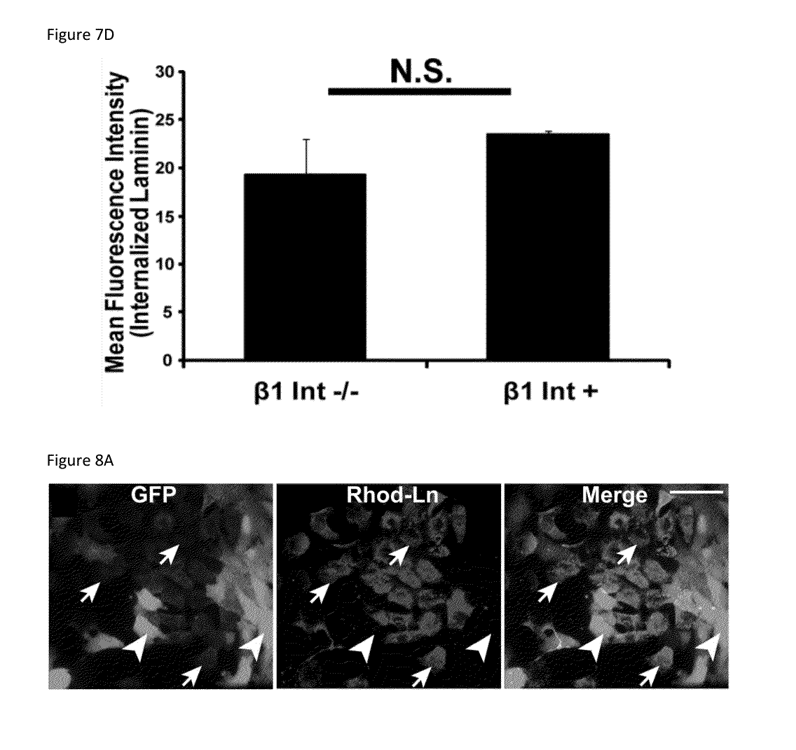

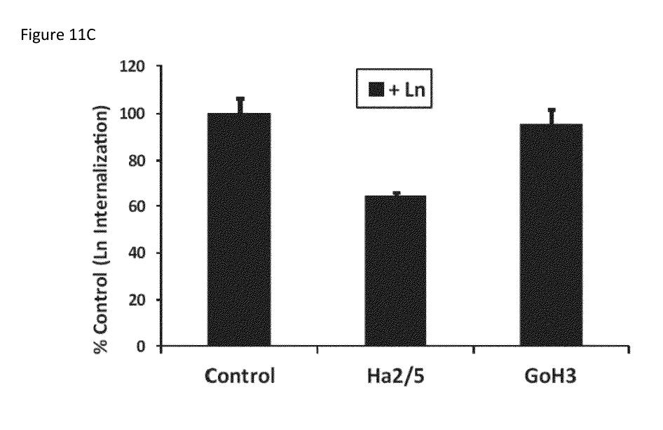

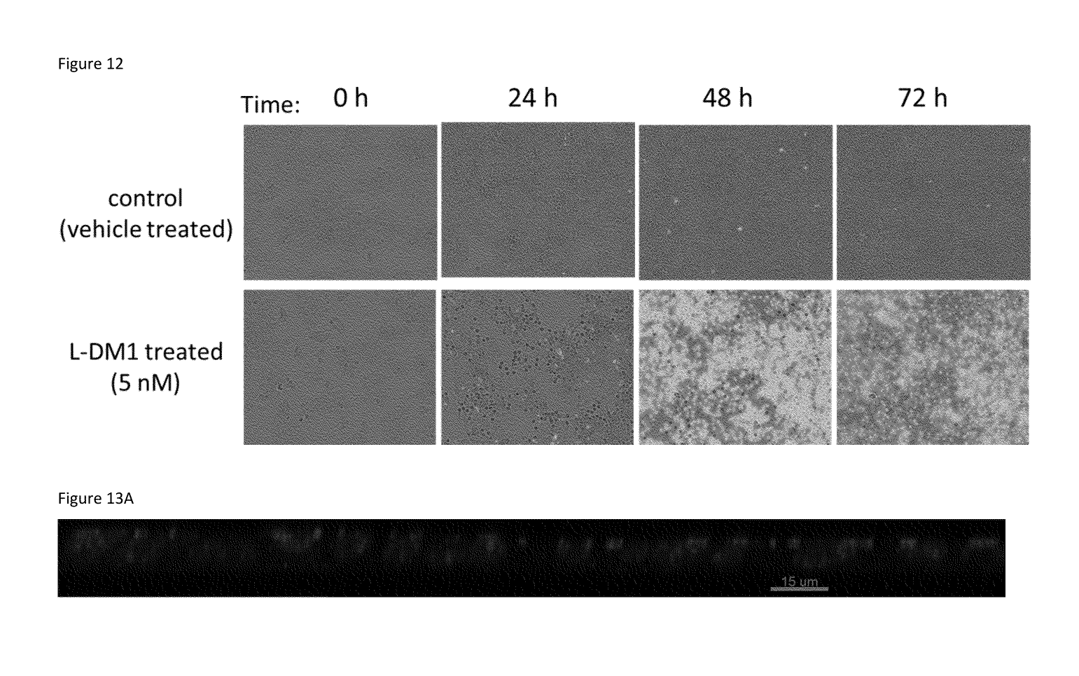

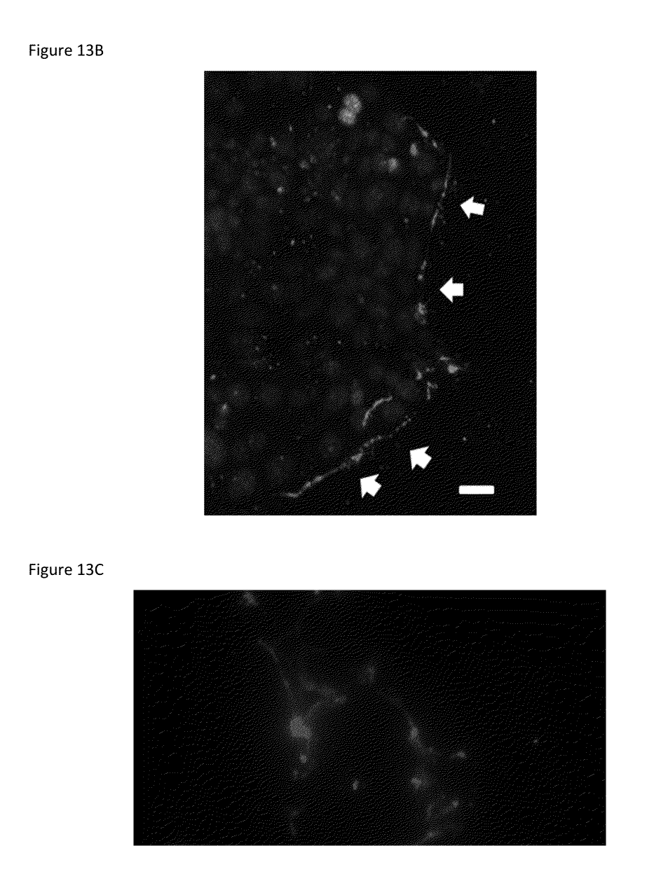

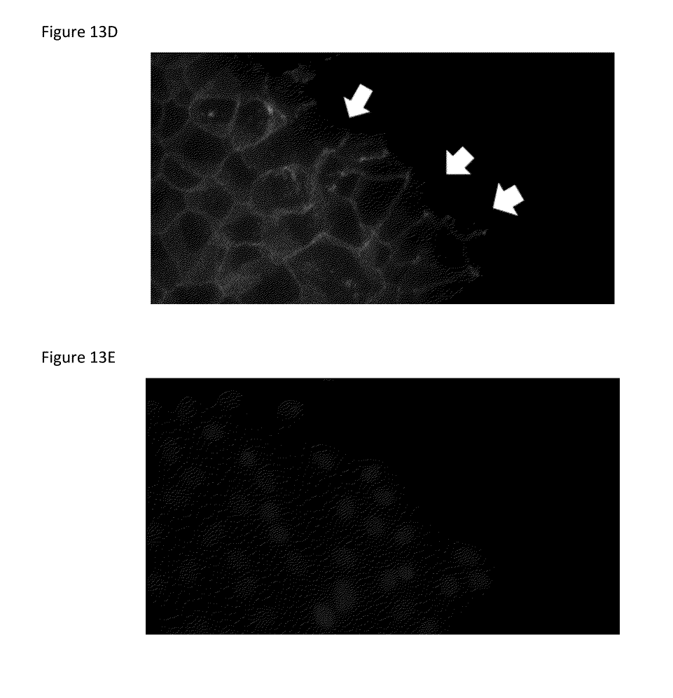

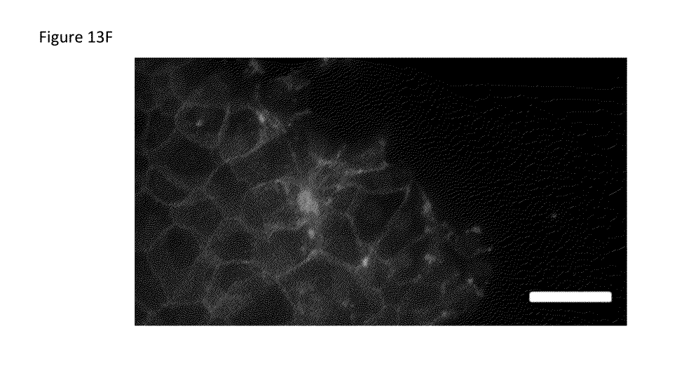

Genetic manipulation of laminin receptor expression was employed in order to identify the specific receptor(s) mediating laminin internalization. The observed inhibition of laminin internalization by heparin (FIG. 6A) suggested dystroglycan (DG) as a mediator because DG binding to the laminin LG4-5 domain is blocked by heparin (Harrison D et al, 2007 supra). To test the role of DG in laminin internalization, an MEC cell line containing an engineered dystroglycan deletion (MEpG) was used (Weir M L et al, 2006 supra). The MEpG cell line was infected with retroviral empty vector (creating control DG^{-/-} cells) or retrovirus expressing DG (creating DG⁺ cells). Immunoblotting confirmed the expected presence or absence of DG expression in these cell types (FIG. 11A). Confocal microscopy and flow cytometry demonstrated a strong reduction in laminin internalization upon deletion of the DG gene which was restored in DG+ cells (FIGS. 7A and 7B). The compiled mean fluorescence intensity (MFI) data of cells expressing DG was nearly four-fold higher than cells lacking DG expression (DG+=18.04±4.6; DG−/−=4.84±1.43, n=6, FIG. 7C). Therefore, the majority of laminin internalization observed in functionally normal mammary epithelial cells appeared to depend on dystroglycan function.

The integrin family of ECM receptors is expressed in the DG^{-/-} cell population (MepG-vec and MepL-vec cell lines, FIG. 11A), but are apparently unable to mediate significant laminin internalization alone (FIG. 7B). To directly test the role of integrins in laminin internalization, an MEC cell line containing an engineered β1 integrin (β1 int) deletion, E3D1cre19 (β1 Int^{-/-}) was used (cre19-vec, FIG. 11A, see Example 10 infra). These β1 int^{-/-} cells were infected with the empty vector retrovirus or with a WT β1 int expressing retrovirus. The re-expression of WT β1 integrin in β1 int^{-/-} cells produced a modest but statistically insignificant change (20% increase) in laminin internalization (MFI β1 Int^{-/-}=21.4±3; β1 Int⁺=26.8±1.2; n=6, p=0.17) (FIG. 7D). A pulsed time course assay showed that the kinetics of laminin internalization was also not altered by β1 integrin expression, although in this assay the magnitude of internalization was moderately lower in β1 Int^{-/-} cells (FIG. 11B). Also, a β1 integrin blocking antibody blocked some 203 laminin internalization in a pulsed internalization assay whereas an α6 integrin blocking antibody showed no effect (FIG. 11C). Therefore, although the β1 integrins are not required for the majority laminin internalization, they can enhance it, possibly as co-receptors with DG (Leonoudakis D et al, 2010 supra; Weir M L et al, 2006 supra). Combined, these data identify DG as the dominant regulator of laminin internalization in functionally normal epithelial cells.

### Example 6—DG is the Dominant Regulator of Both Laminin Assembly and Laminin Internalization, Co Trafficking with Laminin through the Late Endosome

DG has been shown in prior studies to be the dominant regulator of cell-surface laminin assembly (Akhavan A et al, 2012 supra; Leonoudakis D et al, 2010 supra; Weir et al, 2006), and it is surprising that this same receptor should also dominantly regulate laminin internalization.

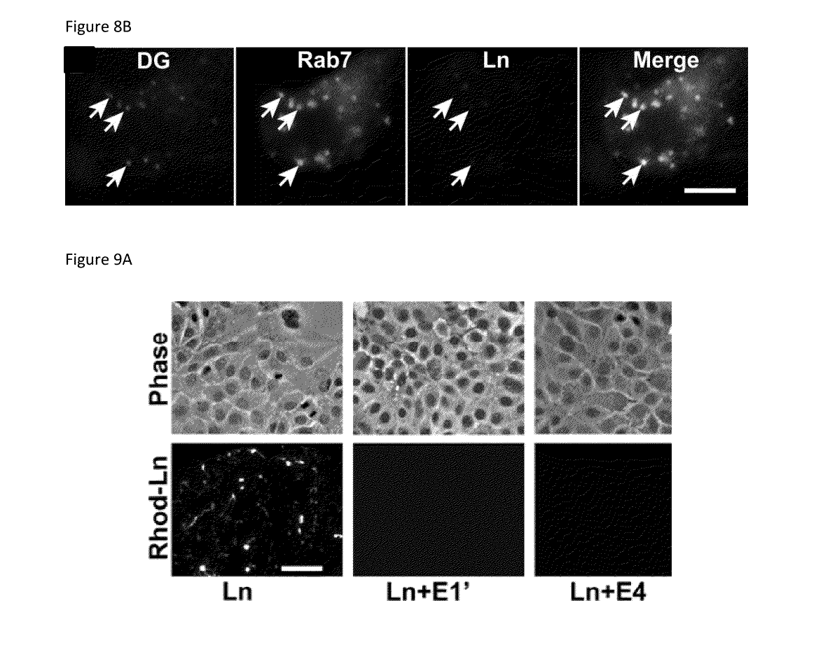

To validate that DG simultaneously and dominantly regulates both laminin assembly and laminin internalization in a cell-autonomous manner, the dynamics of laminin assembly and internalization were assessed via live imaging in co-cultured DG⁺ and DG^{-/-} cells. Both assembly and internalization of laminin was easily and profusely observed in DG⁺ cells, with the internalized laminin visible as rapidly moving vesicles within the cytoplasm (FIG. 8A, arrows). In contrast, both internalization and assembly were undetectable in DG^{-/-} cells during the entire 20 hour time course of the experiment (FIG. 8A arrowheads).

The binding of DG to laminin is a high affinity protein-carbohydrate interaction that persists following laminin internalization indicating that DG may accompany laminin through the protein degradation pathway. Alternatively, their intracellular trafficking patterns may diverge. To distinguish these possibilities, a DG-RFP-encoding fusion construct was co-transfected with the GFP-labeled Rab7 endocytic marker, and these cells treated with Alexa-647 labeled laminin to permit simultaneous tracking of DG and laminin. Live cell imaging of all three proteins showed clear and prominent co-localization of DG and laminin within the late endosome (FIG. 8B). Therefore, DG traffics with laminin through the protein degradation pathway.

### Example 7—Laminin Assembly is not Required for Laminin Endocytosis

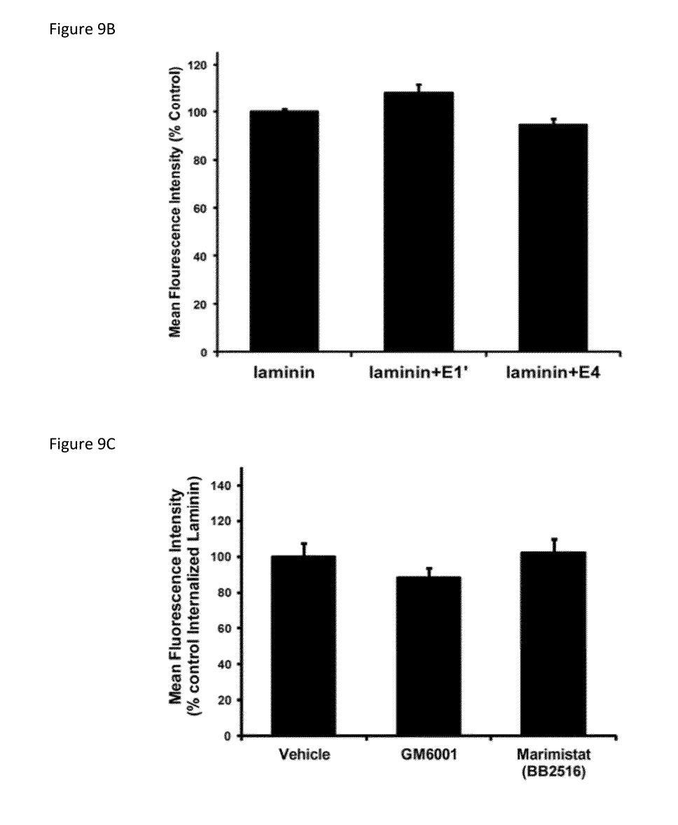

Because DG mediates both assembly and internalization, it was tested whether assembly was required for internalization, employing the E1′ or E4 fragments of laminin which block laminin-111 assembly on myotubes and in MECs (Colognato H et al, J Cell Biol 145, 619-631 (1999) incorporated by reference herein; Weir M L et al, 2006 supra). The laminin E1′ and E4 fragments both blocked assembly of Rho-Ln on the surface of E3D1 MECs (FIG. 9A), however, this blockade of laminin assembly had no effect on the levels of laminin internalization (FIG. 9B). Therefore, laminin assembly is not a prerequisite for laminin internalization, and laminin assembly does not impede internalization, despite both being mediated by the same laminin receptor.

Matrix degradation by the action of proteases could modulate laminin internalization. Matrix metalloproteinase (MMP) activity has been shown to modulate the internalization of fibronectin (Shi F and Sottile J, J Cell Sci 121, 2360-2371 (2008); incorporated by reference herein). Two different broad-spectrum MMP inhibitors GM6001 (50 μM—vehicle=20.2±1.82; GM6001=18.62±1; n=4) and BB2416 (marimistat-5 μM) (vehicle=20.85; BB2416=20.46; n=2) showed no significant effect on steady-state laminin endocytosis in E3D1 MEC cells (FIG. 9C), indicating that MMP activity does not regulate laminin endocytosis.

### Example 8—Loss of DG Function Perturbs LN Internalization in Cancer Cells of Diverse Tissue Origin

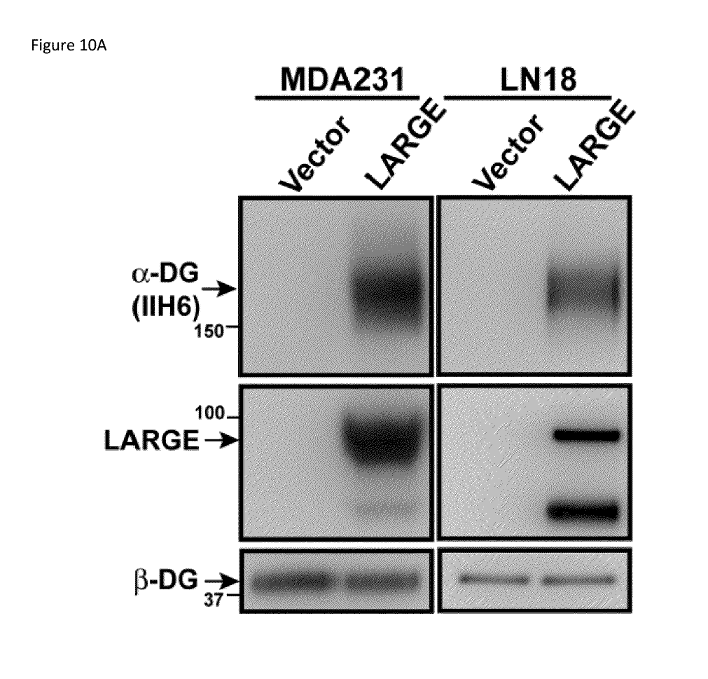

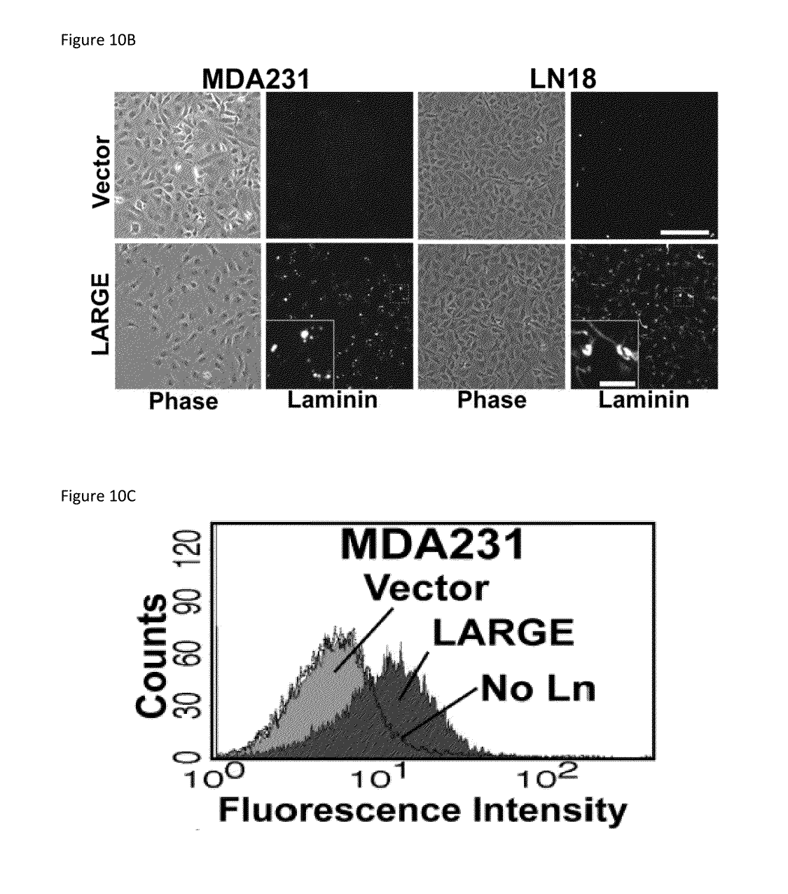

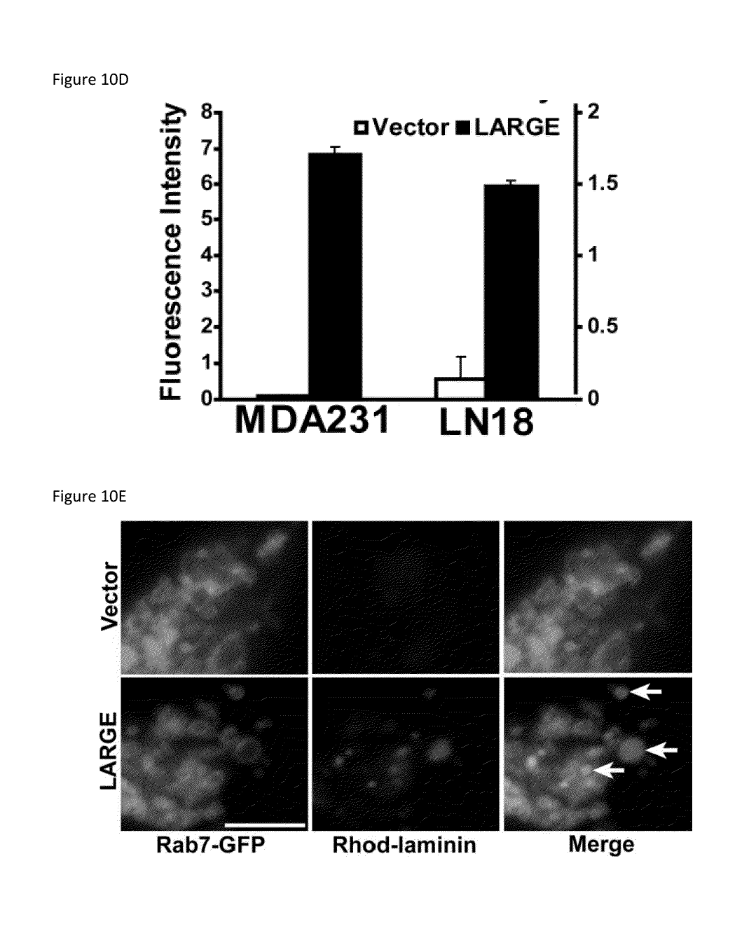

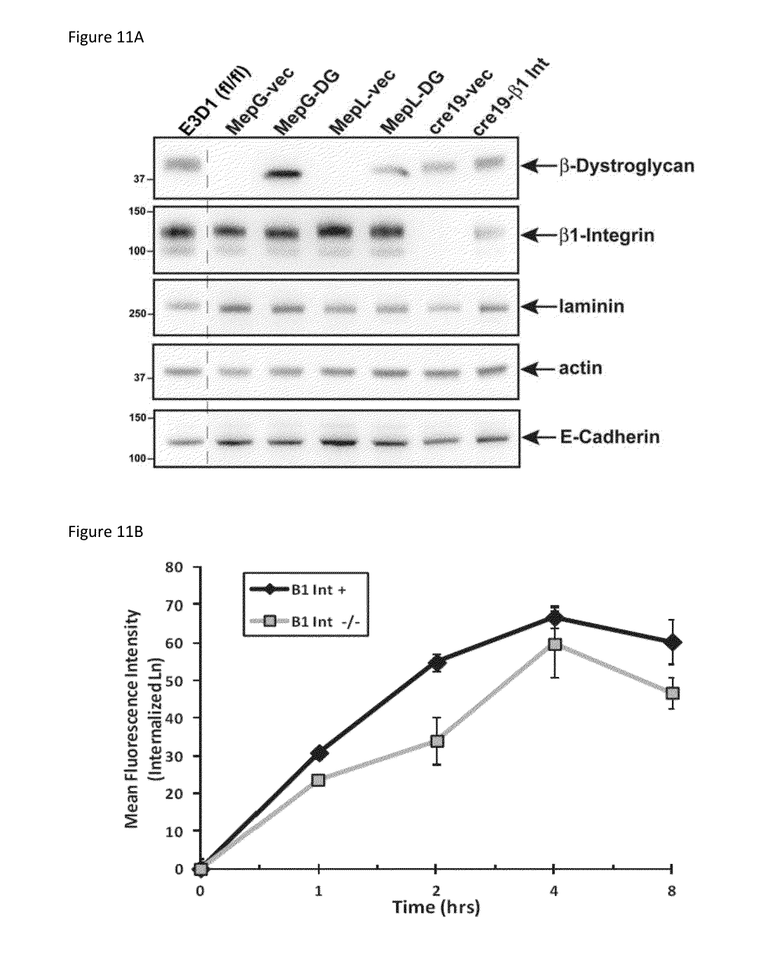

Loss of DG's laminin binding function is a cause of some congenital muscular dystrophies (CMDs) and is a frequent defect in cancers including those of the breast, prostate, colon, and brain (Akhavan A et al, 2012 supra; Beltran-Valero de Bernabe et al, 2009 infra). This loss of function arises from altered glycosylation of DG and can be restored in many carcinoma and glioblastoma cells by expression of the enzyme LARGE, a glycosyltransferase that confers laminin-binding properties to DG (Akhavan et al., 2012; Beltran-Valero de Bernabe D et al, J Biol Chem 284, 11279-11284 (2009); incorporated by reference herein. Based on these facts in light of the disclosure herein, it was hypothesized that laminin internalization would be severely disrupted in cancer cells lacking DG activity and enhanced by the restoration of such activity. This hypothesis was tested in MDA-MB-231 human breast cancer cells and LN18 human glioma cells, both of which lack DG glycosylation and function. Expression of an empty retroviral vector in these cells created the control cells exhibiting the hypoglycosylated DG (FIG. 10A, vector) and lack of laminin assembly at the cell surface (FIG. 10B, vector). Expression of LARGE restored normal glycosylation of DG as determined by western blot analysis with IIH6 antibody (FIG. 10A, LARGE) and functional interaction of DG in the laminin assembly assay (FIG. 10B, LARGE). These cells were subsequently assayed for laminin internalization by flow cytometry. Control cells showed almost no measurable internalization of laminin over background despite the expression of multiple laminin binding integrin receptor subunits; MDA-MB-231 cells express the α1, α2, α3, α6, β1 and β4 integrin subunits, but not δ7, α8 or α9 (Daemen A et al, Genome Biol 14, R110 (2013); incorporated by reference herein). In contrast, LARGE expressing cells showed robust laminin internalization (FIGS. 10C and 10D). Compiled flow cytometry data demonstrates the increase in MFI of internalized laminin in both LARGE expressing MDA-MB-231 (68 fold) and LN18 (10 fold) cells. In addition, restoration of laminin internalization by LARGE expression in cancer cells also restored the trafficking of laminin to Rab7-containing vesicles of the late endosome (FIG. 10E).

### Example 9—Materials and Methods

Cell culture—Primary mammary epithelial cells from control or ΔDG^{K14-Cre} mid-pregnant mice were obtained as previously described (Weir et al, 2006 supra). DG-knockout (MEpG and MEpL cells), and WT E3D1 mammary epithelial cells were established as described previously (Weir et al, 2006 supra) from floxed-DG mice. β1 int-knockout (E3D1 cre19) MECs were established from floxed-β1 int primary MECS, as above. MECs were grown in DME/F12, 2% FBS, 10 μg/ml insulin and 5 ng/ml EGF (BD Biosciences, San Jose, Calif., USA). Human DG, β1 int, or wedge β1 int genes (Luo BH et al, Proc Natl Acad Sci USA 102, 3679-3684 (2005); incorporated by reference herein) were cloned into the retroviral expression vector, pBMN-IRES-PURO as described previously (Weir et al, 2006 supra) and verified by sequencing. Retrovirus was generated using Phoenix-ECO packaging cells grown in DME/H21 (UCSF Cell Culture Facility, San Francisco, Calif., USA) and 10% FBS and transfected using calcium phosphate (Sambrook J et al, Molecular Cloning, a Laboratory Manual, Cold Spring Harbor Laboratory Press, 1989). Clones were seeded into 100 mm dishes, infected with 2 ml of retroviral supernatant, 6 ml of complete media, and 8 μg/ml polybrene, and selected in complete media with 5 μg/ml puromycin (Sigma-Aldrich Corp., St. Louis, Mo., USA). Primary mammary epithelial cells from WT mid-pregnant mice were obtained and cultured as previously described (Weir et al, 2006 supra). Co-culture experiments utilized DG^{-/-} MEpG cells infected with retrovirus to express either control GFP or a full length DG-GFP fusion protein were performed as described previously (Oppizzi M L et al, Traffic 9, 2063-2072 (2008); incorporated by reference herein). To produce astrocyte cultures, P3 mouse cortex was dissociated with papain and plated in DMEM/10% FBS, after one week in culture, flasks were shaken on a rotator to remove microglia and split into 10 cm cell culture dishes, grown to 90% confluency and split into 24 well dishes for experiments. These cultures produced >95% astrocytes as determined by staining with GFAP astrocyte marker antibody.

Laminin labeling—Laminin-111 (1 mg) (Sigma-Aldrich Corp., St. Louis, Mo., USA) was dialyzed twice overnight against 500 ml of PBS with 10 μM CaCl₂. The dialyzed laminin was then reacted with 10 μg NHS-rhodamine, or a 50 fold molar excess of NHS409 CypHer5 (GE) for 2 hr on ice, followed by dialysis twice overnight against 500 ml of PBS with 10 μM CaCl₂.

Live imaging of laminin assembly and internalization—MECs were plated in 35 mm cell culture dishes with cover glass bottoms pre-coated with poly-D-lysine. 10 μg/ml Rhod-Ln was added for 10 min and excess unbound laminin was washed out. Temperature was controlled at 37° C. using a thermoelectric stage and objective warmer (Bioscience Tools, San Diego, Calif., USA). Images were acquired using Nikon Elements software running a Cascade II, QuantEM 512C camera (Photometrics, Tucson, Ariz., USA) at a rate of 1 frame/30s. The co-culture experiment was captured using a Zeiss Axiovert 200 microscope with a Yokogawa spinning disk (Stanford Photonics XR/Mega-10 ICCD and QED InVivo version 3.1.1 software, Palo Alto, Calif., USA).

Laminin assembly—Labeled laminin-111 was prepared as described above. Cells were grown overnight on Nunc Lab-Tek II glass chamber slides (ThermoScientific, Rochester, N.Y., USA). Labeled laminin was added at a 10 μg/ml, incubated overnight, and fixed with paraformaldehyde. For staining of exogenous unlabeled laminin, cells were blocked with 3% BSA/2% goat serum in PBS. Cells were then incubated with anti-laminin primary antibodies (Sigma) followed by anti-rabbit-Cy3 secondary antibodies. Light and fluorescent microscopy was performed on a TE2000 Nikon inverted microscope (Melville, N.Y., USA) with a Photometrics Coolsnap HQ CCD camera (Tucson, Ariz., USA) controlled with Nikon Elements software.

GFP labeled Vesicle expression—cDNA expression constructs of GFP-tagged Rab proteins, Rab5a, Rab5Q79L, and Rab7 and Rab11a were obtained. Cell lines exhibiting laminin trafficking were transiently transfected with GFP-Rab expression constructs using Lipofectamine (Invitrogen), allowed two days for transgene expression, and exposed to labeled laminin for between 4 and 24 hours prior to imaging. Lamp1-GFP expression was performed using the CellLight Lysosomes-GFP BacMam 2.0 expression system (Life Technologies, Grand Island, N.Y., USA). Cells were imaged 18 hrs following transduction.

Flow Cytometry—Cells were plated in 12 well plates at 200,000 cells/well. The following day, media was changed to serum-free media with or without 10 μg/ml rhodamine laminin or 40 μg/ml FITC-dextran (500S-Sigma-Aldrich Corp., St. Louis, Mo., USA). Unless otherwise indicated, cells were incubated 18-24 hrs, washed once with PBS, and cells trypsinized. Cells were washed in 5 ml cold PBS/1 mM EDTA, pelleted, and resuspended in 1 ml PBS/1 mM EDTA. Using a BD FACScan flow cytometer (BD Biosciences, San Jose, Calif., USA), 10,000 cells/well were counted, background fluorescence from the cell counts with no added laminin subtracted and the mean fluorescence intensity values reported. Graphed data are compiled from duplicate wells from each experiment with a minimum of three separate experiments, unless indicated otherwise.

Immunofluorescent microscopy of internalized laminin—10 μg/ml rhodamine-laminin or unlabeled laminin (Sigma-Aldrich Corp., St. Louis, Mo., USA) was added to cells in serum free media overnight. Cells were then prepared as for flow cytometry. Cells were re-plated on Lab-Tek II glass chamber slides and allowed to adhere for 2-3 hrs followed by fixation with 4% PFA. Cells were stained with the membrane marker FITC458 concanavalin A for 1 hr, washed three times with PBS, and mounted with Fluoromount G (Electron Microscopy Sciences, Hatfield, Pa., USA). Confocal images were acquired with a Nikon C1 laser scanning confocal attached to a Nikon TE2000 inverted microscope (Melville, N.Y., USA).

Biochemistry/SDS-PAGE—Cells were lysed in RIPA lysis buffer (50 mM Tris pH 8.0, 1% NP-40, 0.5% deoxycholate, 0.1% SDS, 1 mM EDTA, 1 mM EGTA 1 mM PMSF, 50 mM NaF, 100 mM Na4P₂O₇, 10 mM Na β-glycerophosphate, 1 mM Na₃VO₄, 1X protease inhibitor cocktail-EMD Chemicals, Philadelphia, Pa., USA) and protein concentration quantified with the DC protein assay (Bio-Rad). 10 μg of extracted proteins were resolved on SDS-PAGE gels, transferred to PVDF membranes (Immobilon-P) (EMD Millipore, Billerica, Mass., USA), and immunoblotted as described (Weir et al., 2006). The following primary antibodies were used for immunoblotting: 1:5000 rabbit anti-actin (Sigma-Aldrich Corp., St. Louis, Mo., USA), 1:2000 mouse E-cadherin, 1:1000 mouse β1 integrin (BD Biosciences, San Jose, Calif., USA), 1:2000 mouse β-DG (MANDAG-2, Developmental Studies Hybridoma Bank, Iowa, USA), and 1:1000 IIH6 mouse α-DG IgM (EMD Millipore, Billerica, Mass., USA). HRP-conjugated secondary antibodies specific for rabbit and mouse IgG were used at 1:10,000; anti-IgM-HRP was used at 1:1000 (Jackson Immunoresearch, West Grove, Pa., USA). Immunoblot signals were visualized by enhanced chemiluminescence (Super Signal West Femto-ThermoScientific, Rockford, Ill., USA) and digitally imaged with an Alpha Innotech imager (San Leandro, Calif., USA). Figures are inverted images processed with Adobe Photoshop.

Statistics—Populations are described as mean +/− s.e.m. and statistical significance determined by the paired Student's t-test (two populations).

### Example 11—Detection and Treatment of Bladder Cancers Through Targeting Altered Tissue Architecture

Described herein is an affinity-based targeting agent for the detection and treatment of early stage bladder cancers. Bladder cancers account for 7% of all new cancers and 3% of cancer deaths in the US. Currently, poor detection and treatment options lead to high recurrence rates, high treatment costs, and poor patient outcomes. An important opportunity for improved bladder cancer treatment lies in the development of reagents that are selectively bound and internalized by bladder cancer cells when administered directly into the bladder.

ECM protein internalization can be exploited for the selective delivery of imaging and therapeutic agents to bladder cancers. This can be achieved by 1) establishing a strong pre-clinical mouse bladder cancer model and 2) applying this model to measure the selective targeting of bladder cancers, in vivo, using fluorescently labeled ECM-derived proteins. Two key unmet needs in bladder cancer management are: 1) the more effective detection, diagnosis, and surveillance of bladder cancers; and 2) the more effective treatment of non-invasive disease to limit recurrence and progression. It well is recognized that bladder cancer detection and treatment can be greatly enhanced by the development of reagents that are selectively internalized by early stage bladder cancer lesions. These can take the form of affinity reagents such as immune-targeted contrast agents and therapeutics. Early stage bladder cancers are particularly amenable to affinity-based immmunotherapies and immunodiagnostics because these reagents can be introduced into the bladder directly (known as “intravesicular” delivery) to target the cancer without the need for systemic exposure to these agents. Therefore, a strong opportunity for improved bladder cancer treatment lies in the development of new intravesicular affinity reagents that are effective at selectively binding bladder cancer cells in vivo and internalizing imaging and/or therapeutic compounds.

As described above, the ECM receptor dystroglycan (DG) offers new methods useful in the selective targeting of reagents to cancers. ECM receptors are confined to the basal cell surface in normal epithelia, but redistributed in cancerous tissue upon loss of polarity. Consequently, this pathway offers unique opportunities for targeting the loss of tissue architecture in cancers where the apico-basal is compromised. Immunotoxins currently in development target cancer cells based principally on protein over-expression. Consequently, these existing reagents are not specific to the cancer alone, and are prone to off target effects and resistance based on the absence of the target. In contrast, the compositions described herein target cancers by the characteristic changes in tissue architecture that accompany cancer progression, not by changes in gene expression. If effective for both cancer detection and treatment, these reagents will also represent the first “theranostic” for bladder cancers.

Preclinical testing can be performed in an animal model of bladder cancer where normal tissue architecture remains intact, and cancers are focal in origin. In particular, a strong pre-clinical mouse bladder cancer model can be established and optimized and this model can be applied to measure the selective targeting of bladder cancers, in vivo, using labeled ECM-derived proteins.

One example of such a pre-clinical model is a previously established mouse bladder cancer model wherein Cre-lox DNA recombination is used to eliminate the PTEN and p53 tumor suppressors at focal points within the bladder epithelium. In this model, transgenic mice are used that carry flanking lox (“floxed”) DNA sequences at the PTEN and p53 tumor suppressor gene loci. Cre-recombinase activity is directed specifically to a subset of cells in the bladder epithelium by direct exposure of the bladder lumen (by catheterization) to a replication defective adenovirus (Adeno-Cre) expressing the Cre recombinase gene (Kasman, L and. Voelkel-Johnson C, J Vis Exp, 82, 10.3791/50181 (2013); incorporated by reference herein). The bladders of the transgenic mice bearing homozygous floxed p53 and PTEN loci (PTEN^{fl/fl} and P53^{fl/fl} loci) are exposed to the Ad-Cre virus at 6 weeks of age. These Adeno-Cre-treated PTEN^{fl/fl}/P53^{fl/fl} mice develop a nonmuscle-invasive carcinoma of the bladder within 6 weeks of injection, and these lesions ultimately progress to muscle-invasive bladder cancer. Importantly, this method of tumorigenesis produces focal cancers as the result of adenovirus infection, with entirely normal tissues adjacent to the transformed cells, and they effectively recapitulate the development and progression of human bladder cancer Puzio-Kuter AM, et al, Genes Dev, 23 675-680 (2009) and Seager C M et al, Cancer Prey Res (Phila) 2 1008-1014 (2009); both of which are incorporated by reference herein).

The pre-clinical mouse cancer model can result in the direct testing of test compounds for the selective targeting of bladder cancer cells in vivo. Mice at 6 weeks post Adeno-Cre exposure can be used for reagent testing because pre-invasive legions are evident at that stage. Targeting assays can be used to test multiple ECM proteins with a focus on laminins, which we have firmly established to be rapidly internalized into cell through binding the receptor dystroglycan. Each ECM protein can be fluorescently labeled for the purpose of tracking protein internalization.

Cancer-bearing mice can be treated intravesicularly with the fluorescently labeled ECM components by the same method used as described above to introduce the Adeno-Cre virus. Subsequently, the mice are be euthanized and bladders removed to test for the selective incorporation of the labeled protein into the bladder cancer cells. Internalization of the fluorescently labeled ECM proteins can be assessed by any method. Examples of such methods include visual assessment by fluorescence microscopy; and quantitative assessment by flow cytometry.

As described above, it is anticipated that the fluorescently labeled ECM protein laminin will be internalized at high levels in the carcinoma cells of the bladder, and at undetectable levels in the normal bladder epithelium.

Each ECM component showing selective cancer targeting activity in the initial testing will be analyzed to determine optimal targeting conditions. Recombinant versions of each targeting agent can be generated and tested with the goal of optimizing large scale production as well as effectiveness. Targeting agents can also be assessed using MRI imaging in the animal model described above to demonstrate effectiveness by non-invasive imaging methods.

The most effective targeting agents identified through imaging assays can be coupled to a cytotoxin or other therapeutic compound and applied in intravesicular treatment of bladder cancers in a mouse model. The Cre-activated mouse model described above progresses to invasive disease, and serves as an excellent model for the testing of therapeutic agents. In short, through pre-clinical testing in animals, we will advance these discoveries as rapidly as possible into clinical trials, both for imaging and treatment.

1. A method of identifying a cell as lacking apico-basal polarity, the method comprising:
contacting the cell with a reagent that specifically binds dystroglycan or a homolog thereof, the reagent further comprising a label;
observing assembly of the label on the cell surface or internalization of the label into acidic vesicles;
wherein assembly of the label or internalization of the label into the cell is an indication that the cell lacks apico-basal polarity.

2. The method of claim 1 wherein the reagent comprises a recombinantly produced dystroglycan binding fragment or domain of laminin, perlecan, agrin, pikachurin, biglycan, or a monoclonal antibody that binds dystroglycan or an antigen binding fragment thereof.

3. The method of claim 2 wherein the reagent comprises SEQ ID NO: 2, SEQ ID NO: 3, or SEQ ID NO: 4.

4. The method of claim 1 wherein the label comprises a fluorescent tag, a radioactive isotope, or a magnetic resonance imaging contrast reagent,

5. The method of claim 4 wherein the assembly or internalization of the label is observed using flow cytometry or magnetic resonance imaging.

6. The method of claim 1 wherein the cell is a cancer cell.

7. The method of claim 6 wherein the cancer cell is a breast cancer cell, a glioblastoma cell, a lung cancer cell, a colon cancer cell, a skin cancer cell, or a bladder cancer cell.

8. The method of claim 1 wherein the contacting the cell occurs within a subject.

9. A method of targeting a payload molecule to a cell, the method comprising:
contacting the cell with a protein that specifically binds dystroglycan or a homolog thereof, the wherein the protein is conjugated to a payload molecule, and wherein the payload molecule slows the growth of the cell, provided that the cell lacks apico-basal polarity.

10. The method of claim 9 wherein the payload molecule comprises a radionuclide, a toxin, a nanoparticle, an siRNA, a protein toxin, or a small molecule drug.

11. The method of claim 9 wherein the cell is derived from lung, breast, brain, colon, bladder, or skin.

12. The method of claim 11 wherein the cell is a lung carcinoma, breast carcinoma, glioblastoma, colon carcinoma, bladder carcinoma, or skin carcinoma.

13. The method of claim 9 wherein the cell is within a subject.

14. A pharmaceutical composition comprising:
a protein that specifically binds dystroglycan and a payload molecule conjugated to the protein.

15. The composition of claim 14 wherein the protein comprises a laminin or any dystroglycan binding mutant or fragment thereof, or and wherein the payload molecule comprises mertansine.

16. A method of identifying a test compound that promotes apico-basal polarity, the method comprising:
contacting a cell with the test compound, wherein the cell lacks apico-basal polarity;
contacting the cell with a reagent that specifically binds dystroglycan or a homolog thereof, the reagent comprising a fluorescent label;
wherein a lack of assembly of the fluorescent label on the cell surface and a lack of internalization of the fluorescent label into acidic vesicles is an indication that the test compound promotes apico-basal polarity.

17. The method of claim 16 wherein the test compound comprises a small molecule, monoclonal antibody, or recombinant polypeptide.
